SEQUENCE LISTING (32 sequences)

SEQ ID NO: 1 - DNA, 2557 bp, Artificial
  Expression Vector for Clostridia species
gtcgcgctggagaccctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtgagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggacaccacaacggtttccctctagaaataattttgtttaactttaagaaggagaggtctcttcatggaacttaataatgtaatattggaaaaagaaggaaaagtagctgtagtaacgattaatagacccaaagcattaaatgcattaaattcagatactttaaaagaaatggattatgttataggtgaaatagaaaatgattcagaagtacttgcagttatacttacaggtgcgggagagaaaagctttgttgcaggagctgacatatcggaaatgaaggaaatgaatactattgaaggtagaaaatttggcatactaggtaataaagtgtttagaaggttggaattgcttgaaaagccagtaattgctgcagttaatggatttgcacttggcggcggctgtgagatagctatgtcttgcgatataagaatcgcatcttcaaatgcaagatttggacagcctgaagttggattaggtattacaccagggtttggcggcactcagagattatctagattagtaggtatgggaatggctaagcaacttatatttacagcacaaaatataaaggcagatgaagctttaagaataggacttgtaaataaagtagtagaaccttctgaattaatgaatactgcaaaagaaatagctaataaaatagtctctaatgcaccagtggcagttaaattatcaaaacaagcaataaatagaggtatgcaatgtgacatagatacggcacttgctttcgaatcagaagcatttggtgaatgcttctctactgaagaccaaaaagatgctatgacagcatttattgaaaaacgaaagattgaaggattcaaaaatagataa

SEQ ID NO: 2 - DNA, 3789 bp, Artificial
  Expression Vector for Clostridia species
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacgcgttagaatactcaagctatgcatcaagcttggtaccgagctcggatccactagtaacggccgccagtgtgctggaattcaggcgaggtctcccgctaataagaagaagtgtgaaaaagcgcagctgaaatagctgcgcttttttgtgtcataaggcgcgctcactatctgcggaacctgcctccttatctgataaaaaatattcgctgcatctttgacttgttattttctttcaaatgcctaatggaattgtgagcggataacaattaattatcttttaaaattataacaaatgtgataaaatacaggggatgaaaacattatctaaaaattaaggaggtgtttctaatgagagaccgtcctgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccagtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 3 - DNA, 4737 bp, Artificial
  Expression Vector for Clostridia species
atggattttaatttaacaagagaacaagaattagtaagacagatggttagagaatttgctgaaaatgaagttaaacctatagcagcagaaattgatgaaacagaaagatttccaatggaaaatgtaaagaaaatgggtcagtatggtatgatgggaattccattttcaaaagagtatggtggcgcaggtggagatgtattatcttatataatcgccgttgaggaattatcaaaggtttgcggtactacaggagttattctttcagcacatacatcactttgtgcttcattaataaatgaacatggtacagaagaacaaaaacaaaaatatttagtacctttagctaaaggtgaaaaaataggtgcttatggattgactgagccaaatgcaggaacagattctggagcacaacaaacagtagctgtacttgaaggagatcattatgtaattaatggttcaaaaatattcataactaatggaggagttgcagatacttttgttatatttgcaatgactgacagaactaaaggaacaaaaggtatatcagcatttataatagaaaaaggcttcaaaggtttctctattggtaaagttgaacaaaagcttggaataagagcttcatcaacaactgaacttgtatttgaagatatgatagtaccagtagaaaacatgattggtaaagaaggaaaaggcttccctatagcaatgaaaactcttgatggaggaagaattggtatagcagctcaagctttaggtatagctgaaggtgctttcaacgaagcaagagcttacatgaaggagagaaaacaatttggaagaagccttgacaaattccaaggtcttgcatggatgatggcagatatggatgtagctatagaatcagctagatatttagtatataaagcagcatatcttaaacaagcaggacttccatacacagttgatgctgcaagagctaagcttcatgctgcaaatgtagcaatggatgtaacaactaaggcagtacaattatttggtggatacggatatacaaaagattatccagttgaaagaatgatgagagatgctaagataactgaaatatatgaaggaacttcagaagttcagaaattagttatttcaggaaaaatttttagataatttaaggaggttaagaggatgaatatagttgtttgtttaaaacaagttccagatacagcggaagttagaatagatccagttaagggaacacttataagagaaggagttccatcaataataaatccagatgataaaaacgcacttgaggaagctttagtattaaaagataattatggtgcacatgtaacagttataagtatgggacctccacaagctaaaaatgctttagtagaagctttggctatgggtgctgatgaagctgtacttttaacagatagagcatttggaggagcagatacacttgcgacttcacatacaattgcagcaggaattaagaagctaaaatatgatatagtttttgctggaaggcaggctatagatggagatacagctcaggttggaccagaaatagctgagcatcttggaatacctcaagtaacttatgttgagaaagttgaagttgatggagatactttaaagattagaaaagcttgggaagatggatatgaagttgttgaagttaagacaccagttcttttaacagcaattaaagaattaaatgttccaagatatatgagtgtagaaaaaatattcggagcatttgataaagaagtaaaaatgtggactgccgatgatatagatgtagataaggctaatttaggtcttaaaggttcaccaactaaagttaagaagtcatcaactaaagaagttaaaggacagggagaagttattgataagcctgttaaggaagcagctgcatatgttgtctcaaaattaaaagaagaacactatatttaagttaggagggatttttcaatgaataaagcagattacaagggcgtatgggtgtttgctgaacaaagagacggagaattacaaaaggtatcattggaattattaggtaaaggtaaggaaatggctgagaaattaggcgttgaattaacagctgttttacttggacataatactgaaaaaatgtcaaaggatttattatctcatggagcagataaggttttagcagcagataatgaacttttagcacatttttcaacagatggatatgctaaagttatatgtgatttagttaatgaaagaaagccagaaatattattcataggagctactttcataggaagagatttaggaccaagaatagcagcaagactttctactggtttaactgctgattgtacatcacttgacatagatgtagaaaatagagatttattggctacaagaccagcgtttggtggaaatttgatagctacaatagtttgttcagaccacagaccacaaatggctacagtaagacctggtgtgtttgaaaaattacctgttaatgatgcaaatgtttctgatgataaaatagaaaaagttgcaattaaattaacagcatcagacataagaacaaaagtttcaaaagttgttaagcttgctaaagatattgcagatatcggagaagctaaggtattagttgctggtggtagaggagttggaagcaaagaaaactttgaaaaacttgaagagttagcaagtttacttggtggaacaatagccgcttcaagagcagcaatagaaaaagaatgggttgataaggaccttcaagtaggtcaaactggtaaaactgtaagaccaactctttatattgcatgtggtatatcaggagctatccagcatttagcaggtatgcaagattcagattacataattgctataaataaagatgtagaagccccaataatgaaggtagcagatttggctatagttggtgatgtaaataaagttgtaccagaattaatagctcaagttaaagctgctaataattaagtcggctcggagaccctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtgagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggacaccacaacggtttccctctagaaataattttgtttaactttaagaaggagaggtctcta

SEQ ID NO: 4 - DNA, 4202 bp, Artificial
  Expression Vector for Clostridia species
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacgcgttagaatactcaagctatgcatcaagcttggtaccgagctcggatccactagtaacggccgccagtgtgctggaattcagggaggtctccgctcaatcatactcgaggcgcgcagcctgaatggcgaatggcgctagcataatcaatcgtcccttcgtgtaaacgaaggggcgttttttatttggcgcgtcgttttacaacggagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaaatagggttttaggtaaaattaagttattttaagaagtaattacaataaaaattgaagttatttctttaaggagggaattattcttaagagaccgtcctgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccagtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 5 - DNA, 2944 bp, Artificial
  Expression Vector for Clostridia species
gtgtggagaccctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtgagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggacaccacaacggtttccctctagaaataattttgtttaactttaagaaggagaggtctcacttatgaaagaagtagtaatagcatcggcagtcagaacagcaatcggaagttatggaaaatcacttaaagacgtaccagctgtagatcttggtgccacagctattaaagaggcagtaaaaaaggcaggtataaaaccagaagatgtaaatgaagttatacttggtaacgttttgcaggctggattaggacaaaatcctgcaagacaagcttcttttaaagcaggacttcctgttgaaattcctgctatgactatcaataaagtctgcggatctggtttgagaacagtatcactggcagcacaaataataaaggctggagatgcagatgtaataattgctggcggcatggaaaatatgtctagggcaccttatctggcgaataatgcgcgctggggatataggatgggcaatgcaaaatttgttgatgagatgatcacagatggactatgggatgcattcaatgattatcatatgggaatcacggcagaaaatatagcagaaaggtggaatatatctagagaagaacaagatgaatttgcattagcatcacaaaaaaaagcagaagaagccattaaatctggacagttcaaggacgaaattgttcctgttgtaataaagggaagaaaaggtgagactgtagtagatacagatgagcatccaagatttggatcaactatagagggacttgcaaagcttaagccggcatttaaaaaagatggaactgttacagctggtaatgcatctggactaaatgattgtgctgctgtactcgtaataatgagtgctgaaaaagcaaaggaactaggagtaaaacctcttgctaaaattgtaagttatggaagtgcaggagtagatcctgctataatgggctatggtccattttatgcaactaaggcagcaattgaaaaagcaggatggactgtagatgaattagatcttattgaatcaaacgaagcatttgctgcccaatctttagcagttgccaaagatttaaaatttgatatgaataaggtaaatgtaaatggcggcgctattgccctcggacaccctattggtgcatcaggggcccgtattcttgttacacttgttcatgccatgcaaaaaagggatgcaaagaaaggattagctactctttgtataggcggcggccagggaactgctatattattagaaaaataa

SEQ ID NO: 6 - DNA, 6058 bp, Artificial
  Expression Vector for Clostridia species
tcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctgcaggataaaaaaattgtagataaattttataaaatagttttatctacaatttttttatcaggaaacagctatgaccgcggccgctcactatctgcggaacctgcctccttatctgataaaaaatattcgctgcatctttgacttgttattttctttcaaatgcctaatggaattgtgagcggataacaattaattatcttttaaaattataacaaatgtgataaaatacaggggatgaaaacattatctaaaaattaaggaggtgttactcatagagaccactggatgtcttccggccgctaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggggggcgtaaacgccgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataaatgtcaggctcccttatacacagccagtctgcaggtcgacgaagactagcatggtctccgtgtcatgattgtaacaatttataaataaaaatcaccttttagaggtggtttttttatttataaattagtgtaggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttct

SEQ ID NO: 7 - DNA, 54 bp, Bacillus subtilis
aagaagaagtgtgaaaaagcgcagctgaaatagctgcgcttttttgtgtcataa

SEQ ID NO: 8 - DNA, 45 bp, Bacillus subtilis
ataatcaatcgtcccttcgtgtaaacgaaggggcgttttttattt

SEQ ID NO: 9 - DNA, 52 bp, Lactococcus lactis
aatttataaataaaaatcaccttttagaggtggtttttttatttataaatta

SEQ ID NO: 10 - DNA, 83 bp, Artificial
  Spacer Sequence for Expression Vector for Clostridia species
taatacgactcactatagggacaccacaacggtttccctctagaaataattttgtttaactttaagaaggaggtctcctcatg

SEQ ID NO: 11 - DNA, 85 bp, Artificial
  Spacer Sequence for Expression Vector for Clostridia species
taatacgactcactatagggacaccacaacggtttccctctagaaataattttgtttaactttaagaaggagaggtctcttcatg

SEQ ID NO: 12 - DNA, 84 bp, Artificial
  Spacer Sequence for Expression Vector for Clostridia species
taatacgactcactatagggacaccacaacggtttccctctagaaataattttgtttaactttaagaaggagaagaccatcatg

SEQ ID NO: 13 - DNA, 82 bp, Artificial
  Spacer Sequence for Expression Vector for Clostridia species
taatacgactcactatagggacaccacaacggtttccctctagaaataattttgtttaactttaagaaggaggtctctaatg

SEQ ID NO: 14 - DNA, 84 bp, Artificial
  Spacer Sequence for Expression Vector for Clostridia species
taatacgactcactatagggacaccacaacggtttccctctagaaataattttgtttaactttaagaaggagaggtctctaatg

SEQ ID NO: 15 - DNA, 84 bp, Artificial
  Spacer Sequence for Expression Vector for Clostridia species
taatacgactcactatagggacaccacaacggtttccctctagaaataattttgtttaactttaagaaggaggaagacataatg

SEQ ID NO: 16 - DNA, 84 bp, Artificial
  Spacer Sequence for Expression Vector for Clostridia species
taatacgactcactatagggacaccacaacggtttccctctagaaataattttgtttaactttaagaaggaggtctcacttatg

SEQ ID NO: 17 - DNA, 86 bp, Artificial
  Spacer Sequence for Expression Vector for Clostridia species
taatacgactcactatagggacaccacaacggtttccctctagaaataattttgtttaactttaagaaggagaggtctcacttatg

SEQ ID NO: 18 - DNA, 85 bp, Artificial
  Spacer Sequence for Expression Vector for Clostridia species
taatacgactcactatagggacaccacaacggtttccctctagaaataattttgtttaactttaagaaggagaagactacttatg

SEQ ID NO: 19 - DNA, 190 bp, Artificial
  Modified promoter for fdx gene of Clostridium autoethanogenum
ggccgctcactatctgcggaacctgcctccttatctgataaaaaatattcgctgcatctttgacttgttattttctttcaaatgcctaatggaattgtgagcggataacaattaattatcttttaaaattataacaaatgtgataaaatacaggggatgaaaacattatctaaaaattaaggaggtgtta

SEQ ID NO: 20 - DNA, 477 bp, Artificial
  Modified promoter for pfor gene of Clostridium authethanogenum
ggccgcaaaatagttgataataatgcagagttataaacaaaggtgaaaagcattacttgtattcttttttatatattattataaattaaaatgaagctgtattagaaaaaatacacacctgtaatataaaattttaaattaatttttaattttttcaaaatgtattttacatgtttagaattttgatgtatattaaaatagtagaatacataagatacttaatttaattaaagatagttaagtacttttcaatgtgcttttttagatgtttaatacaaatctttaattgtaaaagaaatgctgtactatttactgtactagtgacgggattaaactgtattaattataaataaaaaataagtacagttgtttaaaattatattttgtattaaatctaatagtacgatgtaagttattttatactattgctagtttaataaaaagatttaattatatacttgaaaaggagaggaatat

SEQ ID NO: 21 - DNA, 562 bp, Artificial
  Modified promoter for wl gene of Clostridium autoethanogenum
ggccgcagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaaatagggttttaggtaaaattaagttattttaagaagtaattacaataaaaattgaagttatttctttaaggagggaattatat

SEQ ID NO: 22 - DNA, 486 bp, Artificial
  Modifed promoter for pta gene of Clostridium autoethanogenum
ggccgcaatatgatatttatgtccattgtgaaagggattatattcaactattattccagttacgttcatagaaattttcctttctaaaatattttattccatgtcaagaactctgtttatttcattaaagaactataagtacaaagtataaggcatttgaaaaaataggctagtatattgattgattatttattttaaaatgcctaagtgaaatatatacatattataacaataaaataagtattagtgtaggatttttaaatagagtatctattttcagattaaatttttgattatttgatttacattatataatattgagtaaagtattgactagcaaaattttttgatactttaatttgtgaaatttcttatcaaaagttatatttttgaataatttttattgaaaaatacaactaaaaaggattatagtataagtgtgtgtaattttgtgttaaatttaaagggaggaaatgaacatgaaac

SEQ ID NO: 23 - DNA, 83 bp, Artificial
  Modified sequence for T7 promoter and ribosome binding site
taatacgactcactatagggagaccacaacggtttccctctagaaataattttgtttaactttaagaaggagatatacatatg

SEQ ID NO: 24 - DNA, 83 bp, Artificial; identical to SEQ ID NO: 10
  Modified sequence for T7 promoter, ribosome binding site, and BsaI site

SEQ ID NO: 25 - DNA, 82 bp, Artificial; identical to SEQ ID NO: 13
  Modified sequence for T7 promoter, ribosome binding site, and BsaI site

SEQ ID NO: 26 - DNA, 84 bp, Artificial; identical to SEQ ID NO: 16
  Modified sequence for T7 promoter, ribosome binding site, and BsaI site

SEQ ID NO: 27 - DNA, 85 bp, Artificial; identical to SEQ ID NO: 11
  Modified sequence for T7 promoter, ribosome binding site, and BsaI site

SEQ ID NO: 28 - DNA, 84 bp, Artificial; identical to SEQ ID NO: 14
  Modified sequence for T7 promoter, ribosome binding site, and BsaI site

SEQ ID NO: 29 - DNA, 86 bp, Artificial; identical to SEQ ID NO: 17
  Modified sequence for T7 promoter, ribosome binding site, and BsaI site

SEQ ID NO: 30 - DNA, 84 bp, Artificial; identical to SEQ ID NO: 12
  Modified sequence for T7 promoter, ribosome binding site, and BbsI site

SEQ ID NO: 31 - DNA, 84 bp, Artificial; identical to SEQ ID NO: 15
  Modified sequence for T7 promoter, ribosome binding site, and BbsI site

SEQ ID NO: 32 - DNA, 85 bp, Artificial; identical to SEQ ID NO: 18
  Modified sequence for T7 promoter, ribosome binding site, and BbsI site

(19) United States
(12) Patent Application Publication
(10) Pub. No.: US 2023/0015505 A1
(43) Pub. Date: Jan. 19, 2023

(54) MODULAR, CELL-FREE PROTEIN EXPRESSION VECTORS TO ACCELERATE BIOLOGICAL DESIGN IN CELLS

(71) Applicants: Northwestern University, Evanston, IL (US); LanzaTech, Inc., Skokie, IL (US)

(72) Inventors: Michael C. JEWETT, Evanston, IL (US); Ashty S. KARIM, Chicago, IL (US); Michael KOEPKE, Skokie, IL (US); Darmawi JUMINAGA, Highland Park, IL (US); Fungmin LIEW, Skokie, IL (US)

(73) Assignees: Northwestern University, Evanston, IL (US); LanzaTech, Inc., Skokie, IL (US)

(21) Appl. No.: 17/782,123

(22) PCT Filed: Dec. 3, 2020
(86) PCT No.: PCT/US2020/063162

§ 371 Date: Jun. 2, 2022

Related U.S. Application Data
(60) Provisional application No. 62/943,036, filed on Dec. 3, 2019.

(51) Int. Cl. C12N 15/74; C12N 15/11; C12N 15/66

(52) U.S. Cl. CPC C12N 15/74; C12N 15/11; C12N 15/66; C12N 2330/51

(57) ABSTRACT

Disclosed are compositions, methods, and kits for performing cell-free protein synthesis (CFPS) and for expressing proteins in cells. Particularly disclosed are vectors comprising Golden Gate sites for cloning, methods for preparing such vectors, and the use thereof for performing CFPS and for expressing proteins in cells such as in naturally occurring or recombinant species of Clostridia, including Clostridium autoethanogenum.

### CROSS-REFERENCE TO RELATED PATENT APPLICATIONS

The present application claims the benefit of priority under 35 U.S.C. 119(e) to U.S. Provisional Application No. 62/943,036, filed on Dec. 3, 2019, the content of which is incorporate herein by reference in its entirety.

### STATEMENT REGARDING FEDERALLY SPONSORED RESEARCH OR DEVELOPMENT

This invention was made with government support under SC0018249 awarded by the Department of Energy. The government has certain rights in the invention.

### BACKGROUND

The present invention generally relates to compositions, methods, and kits for performing cell-free protein synthesis (CFPS) and for expressing proteins in cells. More specifically, the present invention relates to vectors comprising Golden Gate sites for cloning, methods for preparing such vectors, and the use thereof for performing CFPS and for expressing proteins in cells such as in naturally occurring or recombinant species of Clostridia, including Clostridium autoethanogenum.

Cell-free expression of enzymes often requires DNA with minimal regulation while cellular expression can be specific to the chassis organism and contain rather complex architectures. This can be taxing on the research and development involved in prototyping biosynthetic pathways in the cell-free environment before subsequently building pathways in cells. However, modifying cell-free expression vectors can often lead to a dramatic decline in expression capabilities. Herein, we have built cell-free vectors modified for simple cloning into Clostridia expression vectors without inhibiting cell-free expression. These cell-free vectors contain modifications in the 5′ and 3′ ends of the original cell-free vector and allow for quick direct assembly of in vivo expression constructs without lengthy and costly re-synthesis and/or subcloning. The disclosed vectors may be utilized in applications that include, but are not limited to: (i) in vitro study of metabolism; (ii) Biomanufacturing and small molecule production; (iii) Enzyme-expression level prototyping to balance heterologous pathways; (iv) Rapid, high-throughput testing of biosynthetic pathways; (v) Enzyme discovery; (vi) Biosynthetic pathway debugging; (vii) Gas fermentation; and (viii) Engineering clostridia to produce chemicals and advanced bioproducts. The disclosed vectors are advantageous when used in CFPS and in subsequent expression of proteins in cells because they can be utilized to perform both of CFPS and subsequent expression of proteins in cells with less recombinant manipulation thereby reducing the costs involved in biologically designing biological pathways in engineered cells.

### SUMMARY

Disclosed are compositions, methods, and kits for performing cell-free protein synthesis (CFPS) and for expressing proteins in cells. Particularly disclosed are vectors, methods for preparing vectors, and the use thereof for performing CFPS and for expressing proteins in cells such as in naturally occurring or recombinant species of Clostridia, including Clostridium autoethanogenum.

### BRIEF DESCRIPTION OF THE FIGURES

FIG. 1. Illustrative embodiment of the vectors and systems disclosed herein.

FIG. 2. Illustration of various options for designing cell-free expression vectors including a recognition site for BsaI downstream of an ribosome binding site (RBS) to provide a first Golden Gate site (GG1), a third Golden Gate site (GG3), and a fifth Golden Gate site (GG5). A. Option 1: BsaI (downstream of RBS); B. Option 2: BsaI (downstream of RBS but RBS sequence unchanged); and C. Option 3: BbsI (downstream of RBS).

FIG. 3. Cell-free expression of GFP using modified vectors (p111, p212, p314, p431, p532, p634, p751, p852, and p954) versus a control vector (pJL1).

FIG. 4. Assembly of expression pathway using multigene expression vectors as disclosed herein. Components from donor plasmids pDN1-sfGFP, pDN2-Pwl, pDN3-sfGFP, pDN4-Ppfor, and pDN5-buk were excised and assembled into a backbone cell expression plasmid using a Golden Gate protocol.

FIG. 5. Cell expression of components illustrated by fluorescence in 5/15 transformed cells.

FIG. 6. Assembly of three genes using cell-free to Clostridium vector system.

FIG. 7. Assembly of two genes into recipient vector using cell-free to Clostridium vector system.

FIG. 8. Assembly of a single gene into recipient vector using cell-free to Clostridium vector system.

FIG. 9. A framework for a modular ‘cell-free to Clostridium’ vector system that enables seamless assembly of cell-free vectors into a Clostridium expression vector. (a) A schematic representation of how information between in vitro and in vivo needs is used to design DNA sequences, JGI facilities can construct DNA designs, and DNA materials can be used in both in vitro and in vivo experiments. Approximate times are noted for cell-free testing, in vivo construct assembly, and DNA synthesis (for new and old workflows). Costs associated to DNA synthesis are calculated with assumptions of 0.1 USD/bp and 1-3 kb genes. (b) The architecture of the modular vector system is shown. Cell-free vectors are made compatible for assemblies by adding unique overhang (Oy) sites generated from BsaI digests.

FIG. 10. Cell-free expression of Golden Gate compatible vectors is sufficient for prototyping biosynthetic enzymes. (a) A schematic representation of three variants (change in where BsaI sites are located) of each of the three donor plasmids in CFE using a reporter sfGFP is shown. (b) sfGFP concentration was measured by fluorescence at 20 h after cell-free reaction start. Data is shown for n=2 independent experiments with average error. (c) Protein concentration at 20 h for Ptb and Buk enzymes expressed from each of the three donor vectors was measured via C¹⁴-leucine incorporation. Data is shown for n=2 independent experiments with average error. (d) 16 enzymes of interest for acid and alcohol fermentation were codon optimized for C. autoethanogenum and cloned into pD1, pD2, and pD3. Protein expression was measured at 20 h for n=3 independent experiments. All error bars represent 1 standard deviation.

FIG. 11. Golden Gate assembly of a 3-gene construct using compatible cell-free vector system. (a) A schematic representation of our Golden Gate assembly workflow including automated assembly consisting of computational design of plasmids, liquid-handling instructions, plasmid assembly, and plasmid confirmation. (b) PCR confirmation of plasmid assembly in six colonies containing the constructed Clostridium expression vector.

FIG. 12. A modular ‘cell-free to Clostridium’ vector system. (A) Two-part assembly for a single gene insertion. (B) Configuration of donor vectors that enables two-gene insertion using defined Ov sites. (C) Modifying pD1 (or pD2, pD3) to hold more than 1 gene allows for complete assembly of more than three genes using the ‘Cell-free to Clostridium’ vector system. (D) pCExpress can be varied for different assembly types. The key parameters are mentioned here with the full table of variants in Table 3.

FIG. 13. Cell-free expression of E. coli optimized DNA sequences produce more protein than Clostridium optimized sequences. 17 gene sequences were codon optimized for E. coli and C. autoethanogenum and placed in pJL1. Each was expressed in CFE with n=2. Average expression is shown after 20 h with error bars representing average error.

FIG. 14. Cell-free expression of C. autoethanogenum optimized DNA sequences produces active protein. The 16 gene sequences codon optimized for C. autoethanogenum expressed in FIG. 13 using ¹⁴C-leucine incorporation (soluble fractions) were run on SDS PAGE (a) and exposed via autoradiography (b). The PAGE gel and resulting autoradiogram are presented here with molecular weights listed. (c) 12 unique biosynthetic pathways to produce butanoic acid (four enzymatic steps from acetyl-CoA) were built by combining lysates containing expressed enzymes from (a)(b). Error bars represent 1 standard deviation with n=3.

### DETAILED DESCRIPTION

### Definitions and Terminology

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a component” should be interpreted to mean “one or more components” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ′B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Polynucleotides and Synthesis Methods

The disclosed methods, devices, kits, and components may utilize and/or include polynucleotides. The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Regarding polynucleotide sequences, the terms “percent identity” and “identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number (e.g., any of SEQ ID NOs:1-32), or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity relative to a reference sequence (e.g., which is or comprises any of SEQ ID NOs:1-32) over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). For example, a variant, mutant, or derivative of a reference sequence may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length of the reference sequence (e.g., where the reference sequence is or comprises any of SEQ ID NOs:1-32).

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized and/or codon-adapted for expression in a particular host. In the art, codon usage frequency tables have been prepared for a number of host organisms including humans, mouse, rat, pig, E. coli, plants, and other host cells. In some embodiments, the polynucleotide sequences disclosed herein may encode a protein (e.g., a reporter protein such as luciferase) and may be codon-optimized and/or codon-adapted for expression in Clostridia (e.g., Clostridium acetobutylicum, Clostridium autoethanogenum and/or E. coli).

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target,” “target sequence”, “target region”, and “target nucleic acid,” as used herein, are synonymous and refer to a region or sequence of a nucleic acid which is to be amplified, sequenced, or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)ₙ sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, RNA polymerases of bacteriophages (e.g. T3 RNA polymerase, T7 RNA polymerase, SP6 RNA polymerase), and E. coli RNA polymerase, among others. The foregoing examples of RNA polymerases are also known as DNA-dependent RNA polymerase. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, the term “sequence defined biopolymer” refers to a biopolymer having a specific primary sequence. A sequence defined biopolymer can be equivalent to a genetically-encoded defined biopolymer in cases where a gene encodes the biopolymer having a specific primary sequence. As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use as nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

In certain exemplary embodiments, vectors such as, for example, expression vectors, containing a nucleic acid encoding one or more rRNAs or reporter polypeptides and/or proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more rRNAs or reporter polypeptides and/or proteins described herein) in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more rRNAs or reporter polypeptides and/or proteins described herein is linked to the regulatory sequence(s) in a manner which allows for expression of the nucleotide sequence (e.g., in an in vitro ribosomal assembly, transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990).

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed. Vectors as disclosed herein may include plasmid vectors.

Oligonucleotides and polynucleotides may optionally include one or more non-standard nucleotide(s), nucleotide analog(s) and/or modified nucleotides. Examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

Peptides, Polypeptides, Proteins, and Synthesis Methods

The disclosed methods, devices, kits, and components may be utilized to synthesize proteins, polypeptides, and/or peptides. As used herein, the terms “protein” or “polypeptide” or “peptide” may be used interchangeably to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard, noncanonical, or unnatural amino acids, which optionally may include amino acids other than any of the following amino acids: alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine residues. The term “amino acid residue” may include alpha-, beta-, gamma-, and delta-amino acids.

In some embodiments, the term “amino acid residue” may include nonstandard, noncanonical, or unnatural amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard, noncanonical, or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynyl, ether, thiol, sulfonyl, seleno, ester, thioacid, borate, boronate, 17ufa17hor, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

As used herein, a “peptide” is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length ≥100 amino acids (Garrett & Grisham, Biochemistry, 2^{nd} edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

A peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as famesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may include one or more insertions, deletions, or substitution of at least one amino acid residue relative to a reference polypeptide.

Regarding proteins, a “deletion” refers to a change in the amino acid sequence that results in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number (e.g., a polypeptide sequence encoded by any of SEQ ID NOs:1-32), or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein).

The disclosed proteins may be substantially isolated or purified. The term “substantially isolated or purified” refers to proteins that are removed from their natural environment, and are at least 60% free, preferably at least 75% free, and more preferably at least 90% free, even more preferably at least 95% free from other components with which they are naturally associated.

The proteins disclosed herein may be expressed from a “translation template.” As used herein, “translation template” refers to an RNA product of transcription from an expression template that can be used by ribosomes to synthesize polypeptides or proteins.

The proteins disclosed herein may be expressed in a “reaction mixture.” The term “reaction mixture,” as used herein, refers to a solution containing reagents necessary to carry out a given reaction. A reaction mixture is referred to as complete if it contains all reagents necessary to perform the reaction. Components for a reaction mixture may be stored separately in separate container, each containing one or more of the total components. Components may be packaged separately for commercialization and useful commercial kits may contain one or more of the reaction components for a reaction mixture.

Cell-Free Protein Synthesis

Cell-free protein synthesis (CFPS) and methods for making cell extracts for use in CFPS are known in the art. (See, e.g., Carlson et al., “Cell-free protein synthesis: Applications come of age,” Biotech. Adv. Vol. 30, Issue 5, September-October 2012, Pages 1185-1194; Hodgman et al., “Cell-free synthetic biology: Thinking outside the cell,” Metabol. Eng. Vol. 14, Issue 3, May 2012, Pages 261-269; and Harris et al., “Cell-free biology: exploiting the interface between synthetic biology and synthetic chemistry,” Curr. Op. Biotech. Vol. 23, Issue 5, October 2012, Pages 672-678; see also U.S. Pat. Nos. 7,312,049; 7,008,651; and 6,994,986; see also U.S. Published Application Nos. 20170306320; 20160362708; 20160060301; 20120088269; 20090042244; 2008024821; 20080138857; 20070154983; 20070141661; 20050186655; 20050148046120050064592; 20050032086; 20040209321; and 20040038332; the contents of which are incorporated herein by reference in their entireties).

The disclosed compositions may include platforms for preparing a sequence defined biopolymer of protein in vitro. The platforms for preparing a sequence defined polymer or protein in vitro comprises a cellular extract from an organism, and in particulara species of Clostridia, such as Clostridium autoethanogenum. Because CFPS exploits an ensemble of catalytic proteins prepared from the crude lysate of cells, the cell extract (whose composition is sensitive to growth media, lysis method, and processing conditions) is an important component of extract-based CFPS reactions. A variety of methods exist for preparing an extract competent for cell-free protein synthesis, including those disclosed in U.S. Published Application No. 20140295492, published on Oct. 2, 2014, which is incorporated by reference.

The platform may comprise an expression template, a translation template, or both an expression template and a translation template. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. In certain embodiments the platform comprises both the expression template and the translation template. In certain specific embodiments, the platform may be a coupled transcription/translation (“Tx/Tl”) system where synthesis of a translation template and a sequence defined biopolymer occurs in the same cellular extract.

The platform may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

The platform may comprise an orthogonal translation system. An orthogonal translation system may comprise one or more orthogonal components that are designed to operate parallel to and/or independent of the organism's orthogonal translation machinery. In certain embodiments, the orthogonal translation system and/or orthogonal components are configured to incorporation of unnatural amino acids. An orthogonal component may be an orthogonal protein or an orthogonal RNA. In certain embodiments, an orthogonal protein may be an orthogonal synthetase. In certain embodiments, the orthogonal RNA may be an orthogonal tRNA or an orthogonal rRNA. An example of an orthogonal rRNA component has been described in U.S. Published Application Nos. 20170073381 and 20160060301, the contents of which are incorporated by reference in their entireties. In certain embodiments, one or more orthogonal components may be prepared in vivo or in vitro by the expression of an oligonucleotide template. The one or more orthogonal components may be expressed from a plasmid present in the genomically recoded organism, expressed from an integration site in the genome of the genetically recoded organism, co-expressed from both a plasmid present in the genomically recoded organism and an integration site in the genome of the genetically recoded organism, express in the in vitro transcription and translation reaction, or added exogenously as a factor (e.g., a orthogonal tRNA or an orthogonal synthetase added to the platform or a reaction mixture).

### ILLUSTRATIVE EMBODIMENTS

The following embodiments are illustrative and should not be interpreted to limit the scope of the claimed subject matter.

Embodiment 1. A system comprising one or more of the following components: (a) a backbone vector for insertion of a donor sequence from one or more donor vectors, the backbone vector comprising from 5′→3′: (i) a promoter for expressing a gene of interest in a cell; (ii) a first Golden Gate site for cloning (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); (iii) optionally a counter selectable marker; (iv) a second Golden Gate site for cloning (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); and (v) a transcription termination site; (b) a first donor vector (pDonor1) for cell-free expression of a gene of interest, the pDonor1 comprising from 5′→3′: (i) a promoter for cell-free RNA synthesis; (ii) a first Golden Gate site for cloning (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); (iii) optionally a first gene of interest (Gene1); and (iv) a second Golden Gate site for cloning (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); wherein the optional Gene1 is inserted between the first Golden Gate site and the second Golden Gate site; (c) a second donor vector (pDonor2) that comprises a donor promoter for use in expressing a gene of interest in a cell, the pDonor2 comprising from 5′→3′: (i) a first Golden Gate site for cloning (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); (ii) a transcription termination site; (iii) a promoter for expressing a gene of interest in a cell; and (iv) a second Golden Gate site for cloning (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); and (d) a third donor vector (pDonor3) for cell-free expression of a gene of interest, the pDonor3 comprising from 5′→3′: (i) a promoter for cell-free RNA synthesis; (ii) a first Golden Gate site for cloning (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); (iii) optionally a second gene of interest (Gene2); and (iv) a second Golden Gate site for cloning (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); wherein the optional Gene2 is inserted between the first Golden Gate site and the second Golden Gate site, optionally wherein the system comprises any combination of components selected from: (a) and (b); (a) and (c); (a) and (d); (b) and (c); (b) and (d); (c) and (d); (a), (b), and (c); (a), (b), and (d); (a), (c), and (d); (b), (c), and (d); and (a), (b), (c), and (d).

Embodiment 2. The system of embodiment 1, wherein the system comprises two or more of the components: (a) the backbone vector, (b) the pDonor1, (c) the pDonor2, and (d) the pDonor3.

Embodiment 3. The system of embodiment 1 or 2, wherein the system comprises component (a) the backbone vector; and one or more of components (b) the pDonor1, (c) the pDonor2, and (d) the pDonor3.

Embodiment 4. The system of any of the foregoing embodiments, wherein the system comprises components (a) the backbone vector, (b) the pDonor1, (c) the pDonor2, and (d) the pDonor3.

Embodiment 5. A system comprising one or more of the following components: (a) a backbone vector for insertion of a donor sequence from a donor vector, the backbone vector comprising from 5′→3′: (i) a first promoter for expressing a gene of interest in a cell (P1); (ii) a first Golden Gate site for cloning (GG1) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); (iii) optionally a counter selectable marker; (iv) a second Golden Gate site for cloning (GG2) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); and (v) a transcription termination site (TT); (b) a first donor vector (pDonor1) for cell-free expression of a gene of interest, the pDonor1 comprising from 5′→3′: (i) a promoter for cell-free RNA synthesis; (ii) a first Golden Gate site for cloning (GG1) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); (iii) optionally a first gene of interest (Gene1); and (iv) a second Golden Gate site for cloning (GG2) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); wherein the optional Gene1 is inserted between GG1 and GG2.

Embodiment 6. A system comprising one or more of the following components: (a) a backbone vector for insertion of donor sequences from donor vectors, the backbone vector comprising from 5′→3′: (i) a first promoter for expressing a gene of interest in a cell (P1); (ii) a first Golden Gate site for cloning (GG1) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); (iii) optionally a counter selectable marker, for example, a toxin such as ccdB; (iv) a terminal Golden Gate site for cloning (GGT) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); and (v) a terminal transcription termination site (TT); (b) a first donor vector (pDonor1) for cell-free expression of a gene of interest, pDonor1 comprising from 5′→3′: (i) a promoter for cell-free RNA synthesis (e.g., a promoter for T7 RNA polymerase); (ii) a first Golden Gate site for cloning (GG1) (i.e. a recognition site for a TypeIIS restriction enzyme such as a BsaI site or a BbsI site or an AarI site optionally oriented so as to cleave upstream (5′) of its recognition site and provide an overhang that hybridizes to the overhang of GG1 in the backbone vector; (iii) optionally a first gene of interest (Gene1); and (iv) a second Golden Gate site for cloning (GG2) (i.e. a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to its reverse complement overhang); where the optional Gene1 is inserted between GG1 and GG2; (c) a second donor vector (pDonor2) that comprises a donor promoter for use in expressing a gene of interest in a cell, pDonor2 comprising from 5′→3′: (i) a second Golden Gate site for cloning (GG2) (i.e. a recognition site for a TypeIIS restriction enzyme such as a BsaI site or a BbsI site or an AarI site oriented so as to cleave outside of its recognition site and provide an overhang (e.g., a 5′ overhang) that hybridizes to the overhang of GG2 in pDonor1; (ii) a first transcription termination site (T1); (iii) a second promoter for expressing a gene of interest in a cell (P2); and (iv) a third Golden Gate site for cloning (GG3) (i.e. a recognition site for a TypeIIS restriction enzyme such as a BsaI site or a BbsI site or an AarI site oriented so as to cleave outside of its recognition site and provide an overhang (e.g., a 5′ overhang); and (d) a third donor vector (pDonor3) for cell-free expression of a gene of interest, pDonor3 comprising from 5′→3′: (i) a promoter for cell-free RNA synthesis (e.g., a promoter for T7 RNA polymerase); (ii) a third Golden Gate site for cloning (GG3) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to the overhang of GG3 in pDonor2); (iii) optionally a second gene of interest (Gene2); and (iv) a terminal Golden Gate site for cloning (GGT) (i.e. a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to the overhang of GGT in the backbone vector); where the optional Gene2 is inserted between GG3 and GGT.

Embodiment 7. A system comprising one or more of the following components: (a) a backbone vector for insertion of donor sequences from donor vectors, the backbone vector comprising from 5′→3′: (i) a first promoter for expressing a gene of interest in a cell (P1); (ii) a first Golden Gate site for cloning (GG1) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); (iii) optionally a counter selectable marker, for example, a toxin such as ccdB; (iv) a terminal Golden Gate site for cloning (GGT) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); and (v) a terminal transcription termination site (TT); (b) a first donor vector (pDonor1) for cell-free expression of a gene of interest, pDonor1 comprising from 5′→3′: (i) a promoter for cell-free RNA synthesis (e.g., a promoter for T7 RNA polymerase); (ii) a first Golden Gate site for cloning (GG1) (i.e. a recognition site for a TypeIIS restriction enzyme such as a BsaI site or a BbsI site or an AarI site optionally oriented so as to cleave upstream (5′) of its recognition site and provide an overhang that hybridizes to the overhang of GG1 in the backbone vector; (iii) optionally a first gene of interest (Gene1); and (iv) a second Golden Gate site for cloning (GG2) (i.e. a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to its reverse complement overhang); where the optional Gene1 is inserted between GG1 and GG2; (c) a second donor vector (pDonor2) that comprises a donor promoter for use in expressing a gene of interest in a cell, pDonor2 comprising from 5′→3′: (i) a second Golden Gate site for cloning (GG2) (i.e. a recognition site for a TypeIIS restriction enzyme such as a BsaI site or a BbsI site or an AarI site oriented so as to cleave outside of its recognition site and provide an overhang (e.g., a 5′ overhang) that hybridizes to the overhang of GG2 in pDonor1; (ii) a first transcription termination site (T1); (iii) a second promoter for expressing a gene of interest in a cell (P2); and (iv) a third Golden Gate site for cloning (GG3) (i.e. a recognition site for a TypeIIS restriction enzyme such as a BsaI site or a BbsI site or an AarI site oriented so as to cleave outside of its recognition site and provide an overhang (e.g., a 5′ overhang); and (d) a third donor vector (pDonor3) for cell-free expression of a gene of interest, pDonor3 comprising from 5′→3′: (i) a promoter for cell-free RNA synthesis (e.g., a promoter for T7 RNA polymerase); (ii) a third Golden Gate site for cloning (GG3) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to the overhang of GG3 in pDonor2); (iii) optionally a second gene of interest (Gene2); and (iv) a fourth Golden Gate site for cloning (GG4) (i.e. a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to its reverse complement overhang); where the optional Gene2 is inserted between GG3 and GG4; (e) a fourth donor vector (pDonor4) that comprises a donor promoter for use in expressing a gene of interest in a cell, pDonor4 comprising from 5′→3′: (i) a fourth Golden Gate site for cloning (GG4) (i.e. a recognition site for a TypeIIS restriction enzyme such as a BsaI site or a BbsI site or an AarI site oriented so as to cleave outside of its recognition site and provide an overhang (e.g., a 5′ overhang) that hybridizes to the overhang of GG4 in pDonor3; (ii) a second transcription termination site (T2); (iii) a third promoter for expressing a gene of interest in a cell (P3); and (iv) a fifth Golden Gate site for cloning (GG5) (i.e. a recognition site for a TypeIIS restriction enzyme such as a BsaI site or a BbsI site or an AarI site oriented so as to cleave outside of its recognition site and provide an overhang (e.g., a 5′ overhang) that hybridizes to its reverse complement overhang; and (f) a fifth donor vector (pDonor5) for cell-free expression of a gene of interest, pDonor5 comprising from 5′→3′: (i) a promoter for cell-free RNA synthesis (e.g., a promoter for T7 RNA polymerase); (ii) a fifth Golden Gate site for cloning (GG5) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to the overhang of GG5 in pDonor4); (iii) optionally a third gene of interest (Gene3); and (iv) a terminal Golden Gate site for cloning (GGT) (i.e. a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to the overhang of GGT in the backbone vector); where the optional Gene3 is inserted between GG5 and GGT.

Embodiment 8. The system of any of the foregoing embodiments, wherein pDonor1, pDonor3, pDonor5 comprise a first gene, a second gene, and a third gene respectively, such as Gene1, Gene2, and/or Gene3 respectively, wherein optionally Gene1, Gene2, and/or Gene3 have been codon-optimized for expression in a cell-free system, optionally, a cell-free system comprising a cellular lysate from Clostridia; and/or wherein optionally Gene1, Gene2, and/or Gene3 have been codon-optimized for expression in a cell, optionally a Clostridia cell.

Embodiment 9. The system of any of the foregoing embodiments, wherein pDonor2 and pDonor4 comprise a promoter that has been engineered to express a gene in Clostridia or in a cell-free extract prepared from Clostridia.

Embodiment 10. The system of any of the foregoing embodiments, wherein GG1 is a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang.

Embodiment 11. The system of any of the foregoing embodiments, wherein GG2 is a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to its reverse complement overhang).

Embodiment 12. The system of any of the foregoing embodiments, wherein GG3 is a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to its reverse complement overhang).

Embodiment 13. The system of any of the foregoing embodiments, wherein GG4 is a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to its reverse complement overhang).

Embodiment 14. The system of any of the foregoing embodiments, wherein GG5 is a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to its reverse complement overhang).

Embodiment 15. The system of any of the foregoing embodiments, wherein GGT is a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang).

Embodiment 16. The system of any of the foregoing embodiments, wherein the counter selectable marker is toxin such as ccdB.

Embodiment 17. The system of any of the foregoing embodiments, wherein the promoter for cell-free RNA synthesis is the T7 RNA polymerase promoter.

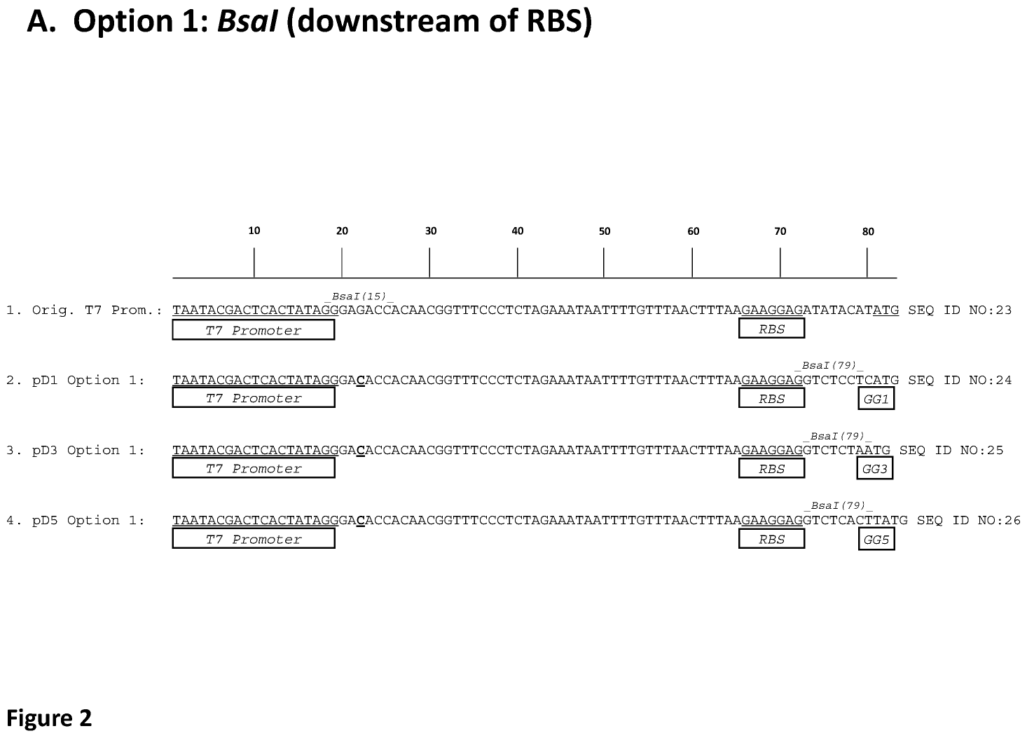

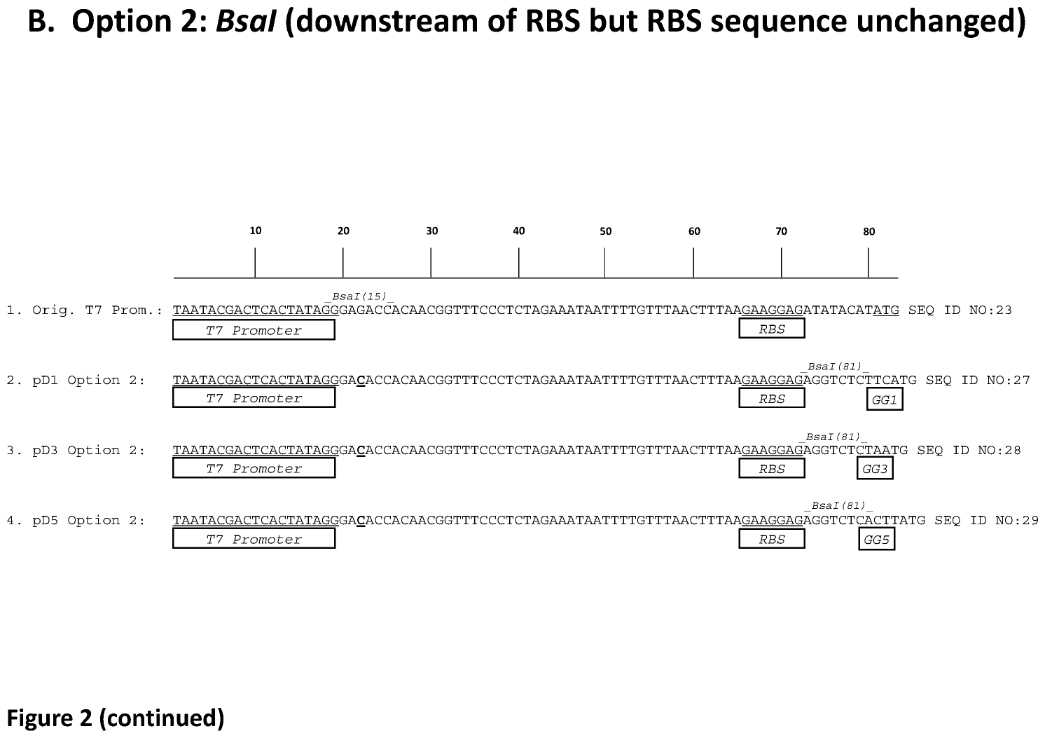

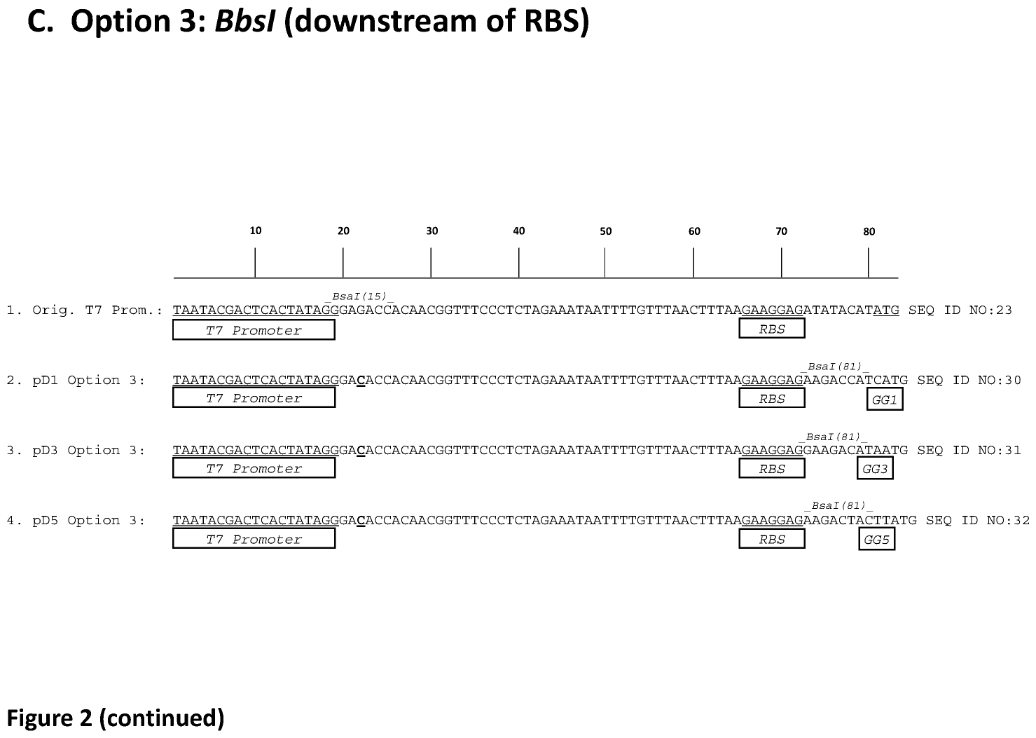

Embodiment 18. The system of any of the foregoing embodiments, wherein: (i) optionally pDonor1 comprises a polynucleotide sequence presented in FIG. 2 and optionally selected from pDonor1 Option 1 (SEQ ID NO:24), pDonor1 Option 2 (SEQ ID NO:27), and pDonor1 Option 3 (SEQ ID NO:30); (ii) optionally pDonor3 comprises a polynucleotide sequence presented in FIG. 2 and optionally selected from pDonor3 Option 1 (SEQ ID NO:25), pDonor3 Option 2 (SEQ ID NO:28), and pDonor3 Option 3 (SEQ ID NO:31); and (iii) optionally pDonor5 comprises a polynucleotide sequence presented in FIG. 2 and optionally selected from pDonor5 Option 1 (SEQ ID NO:26), pDonor5 Option 2 (SEQ ID NO:29), and pDonor5 Option3 (SEQ ID NO:32).

Embodiment 19. A cell transformed with any of the components of the systems of any of the foregoing embodiments.

Embodiment 20. A method for expressing a gene of interest, such as Gene1, Gene2, or Gene3, the method comprising cloning the gene of interest into a vector of any of embodiments 1-14 and expressing the gene of interest in a cell-free system or in a cell (optionally in a cell-free system comprising a cell-free extract prepared from Clostridia cells or in a Clostridia cell).

Embodiment 21. A method for expressing multiple genes of interest in a cell such as Gene1, Gene2, or Gene3, the method comprising cloning the multiple genes of interest into one or more vectors of any of embodiments 1-18, further cloning the multiple genes of interest into the backbone vector of any of embodiments 1-18, introducing the backbone vector into a cell or a cell-free extract and expressing the multiple genes of interest in the cell or in the cell-free extract.

Embodiment 22. The method of embodiment 20 or 21, wherein the multiple genes of interest are expressed from multiple different promoters.

Embodiment 23. A polynucleotide or a combination of two or more polynucleotides, wherein the polynucleotide or polynucleotides comprise one or more polynucleotide sequences selected from SEQ ID NOs:1-32, optionally wherein the polynucleotide or the polynucleotides comprise one or more polynucleotide sequences as presented in FIG. 2, optionally a polynucleotide sequence selected from pDonor1 Option 1 (SEQ ID NO:24), pDonor3 Option 1 (SEQ ID NO:25), pDonor5 Option 1 (SEQ ID NO:26), pDonor1 Option 2 (SEQ ID NO:27), pDonor3 Option 2 (SEQ ID NO:28), pDonor5 Option 2 (SEQ ID NO:29), pDonor1 Option 3 (SEQ ID NO:30), pDonor3 Option 3 (SEQ ID NO:31), and pDonor5 Option 3 (SEQ ID NO:32).

Embodiment 24. A polynucleotide or a combination of two or more polynucleotides, wherein the polynucleotide or the combination of two or more polynucleotides comprises one or more polynucleotides selected from SEQ ID NOs:1-32 and combinations thereof.

### EXAMPLES

The following Examples are illustrative and should not be interpreted to limit the scope of the claimed subject matter.

### Example 1—Modular, Cell-Free Protein Expression Plasmids to Accelerate Biological Design in Cells

Cell-free prototyping of pathways has been shown to inform design and accelerate development cycles (Karim, A. S. & Jewett, M. C. A cell-free framework for rapid biosynthetic pathway prototyping and enzyme discovery. Metabolic Engineering 36, 116-126 (2016)). The cell free framework requires only single genes as input and the produced proteins can then be mixed and matched to identify most promising designs to use in vivo. In contrast in vivo engineering requires genes to be arranged in operons. Yet another challenge is that codon usage of genes is different for each organism.

In a typical workflow one would synthesize genes that are codon-optimized for a cell-free platform, such as an E. coli-derived cell-free platform, then test the genes in the cell-free platform to find the ideal combination of expressed genes to achieve the desired synthesis pathway. This combination of genes would then be codon-optimized for the host of interest (e.g. gas-fermenting Clostridia) and then synthesized and assembled into operons. Cell-free expression is typically done with a T7 promoter whereas in vivo expression is typically achieved using different promoters and each application requires specific vectors (e.g. a vector optimized for cell-free expression and a vector optimized for expression in the target host). This process would typically take 6-9 weeks and cost $600 per gene.

Current Workflow

| Step | Current Workflow | Time | | Cost |
|---|---|---|---|---|
| 1 | Codon-optimize all X genes of interest for E. coli | 1 | day | |
| 2 | Synthesize and clone all X genes into cell-free | 1-2 | weeks | $300 per |
| | expression vector | | | gene |
| 3 | Test genes in cell-free expression system | 1-3 | days | |
| 4 | Codon-optimize all down-selected Y genes of | 1 | day | |
| | interest for Clostridium | | | |
| 5 | Synthesize and clone all down-selected Y genes | 2-3 | weeks | $300 per |
| | into Clostridium expression vector | | | gene |
| 6 | Transform Clostridium with Clostridium | 2-3 | weeks | |
| | expression vector(s) and test production | | | |
| TOTAL | | 6-9 | weeks | $600 per |
| | | | | gene |

Proposed Workflow

| Step | Proposed Workflow | Time | | Cost |
|---|---|---|---|---|
| 1 | Codon-optimize all X genes of interest for | 1 | day | |
| | Clostridium | | | |
| 2 | Synthesize and clone all X genes into modified | 1-2 | weeks | $300 per |
| | cell-free expression vector | | | gene |
| 3 | Test genes in cell-free expression system | 1-3 | days | |
| 4 | Clone all down-selected Y genes from cell-free | 3 | days | $20 per |
| | expression vector into Clostridium expression | | | gene |
| | vector | | | |
| 5 | Transform Clostridium with Clostridium | 2-3 | weeks | |
| | expression vector(s) and test production | | | |
| TOTAL | | 4-6 | weeks | $320 per |
| | | | | gene |

In the proposed scheme, the genes would be codon-optimized for the host of interest (which may have a quite different GC content from E. coli, e.g. Clostridium 30% vs E. coli 50%) then synthesized into a modified cell-free vector that has Golden Gate sites introduced that allow for a direct assembly after the cell-free assessments have been completed. In vivo expression often also requires the use of different promoters in a library, the proposed concept allows for this. This cuts costs and time requirements in half.

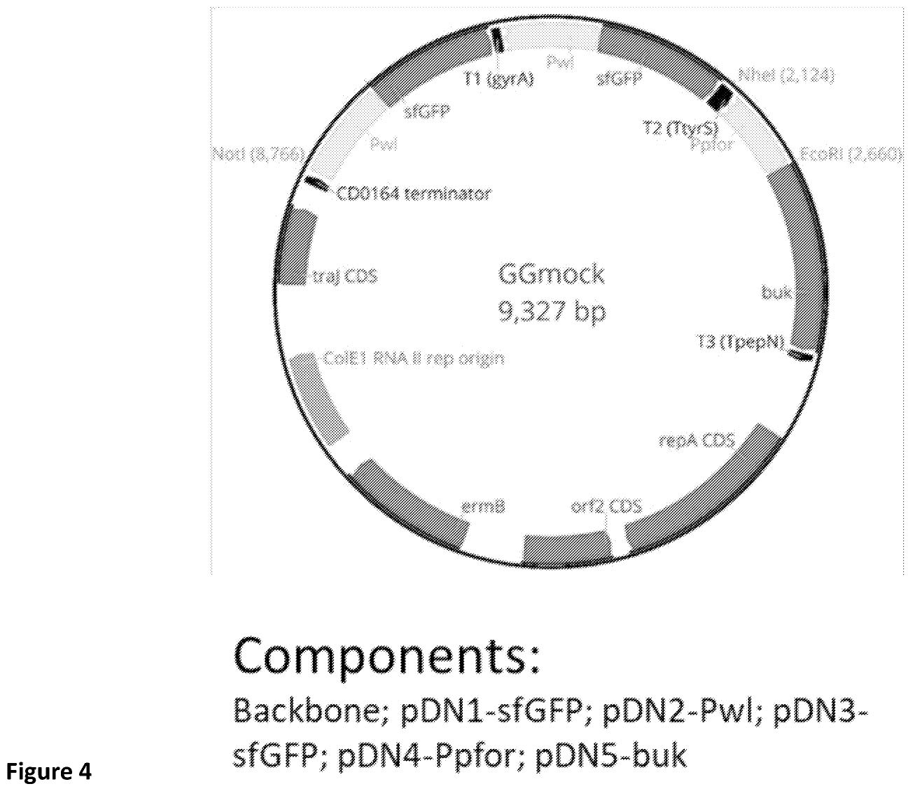

Previously it was found that any changes to the cell free vector affected expression negatively. Surprisingly introduction of Golden Gate sites at different positions around the RBS and promoter regions did not affect cell-free expression negatively via downregulation and in instances even led to higher expression. For efficient assembly of expression vector for in vivo testing in the target host (e.g. Clostridia), Golden Gate cut sites and overhangs were introduced to both the recipient and donor vectors. In one embodiment, the recipient vectors contain Gram positive replicons suitable for plasmid propagation in Clostridia, antibiotic resistance genes and the ccdB counter selectable marker to facilitate efficient screening of assembled constructs. The donor vectors constitute the cell-free expression vector with T7 promoter, or subcloning vector with a terminator and a Clostridial promoter. Two Golden Gate cut sites and appropriate overhangs were introduced into each donor vectors. These modifications occur in the ribosomal binding site (RBS) region and alter the nucleotide length between the RBS and START codon of the T7 promoter in the cell-free vector. To ensure that these modifications would not significantly impact expression, all nine cell-free vectors with modified T7 promoters were used to express sfGFP as a fluorescent reporter gene to measure the activity of the modified T7 promoters. (See FIG. 2 for modified T7 promoter sequences, namely pDonor1 Option 1 (SEQ ID NO:24), pDonor3 Option 1 (SEQ ID NO:25), pDonor5 Option 1 (SEQ ID NO:26), pDonor1 Option 2 (SEQ ID NO:27), pDonor3 Option 2 (SEQ ID NO:28), pDonor5 Option 2 (SEQ ID NO:29), pDonor1 Option 3 (SEQ ID NO:30), pDonor3 Option 3 (SEQ ID NO:31), and pDonor5 Option 3 (SEQ ID NO:32)). Results showed that not only is expression not negatively affected, 8 out of 9 modified T7 promoter actually showed enhanced promoter activity (up to ˜40% improvement) and a 3 gene pathway has been successfully assembled for in vivo expression directly from modified cell-free vectors. (See FIG. 4).

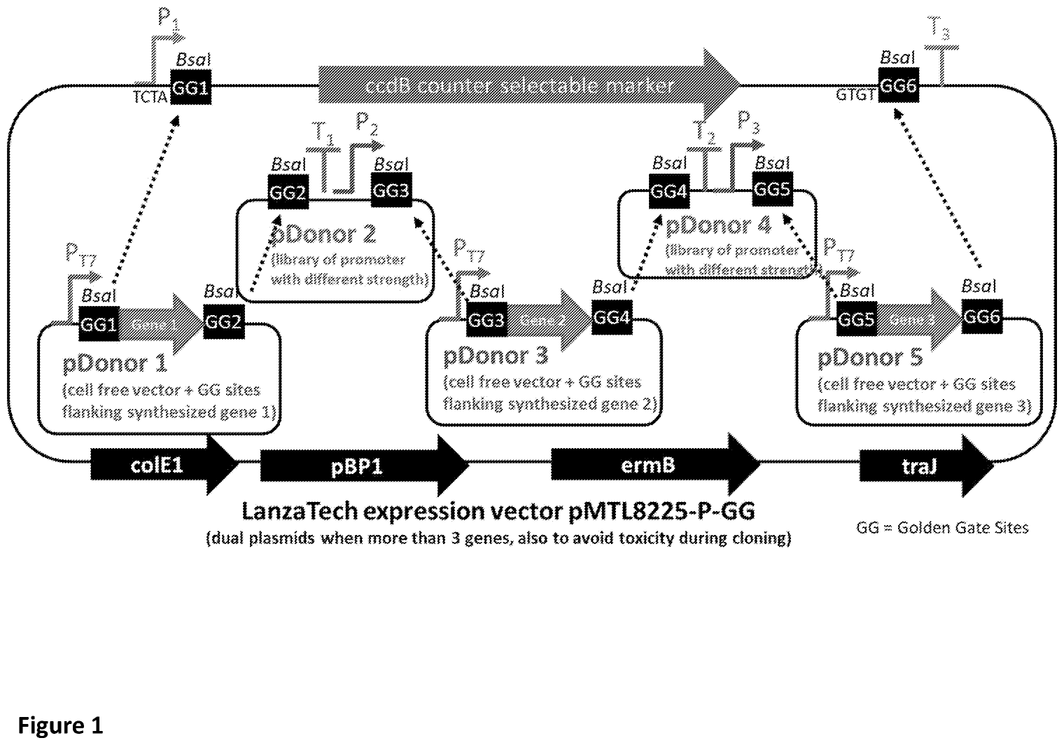

FIG. 1 illustrates one embodiment of the disclosed vectors and systems. As illustrated in FIGS. 1, the LanzaTech expression vector pMTL8225-P-GG is utilized as a backbone vector for insertion of donor sequences from donor vectors. As indicated, pMTL8225-P-GG, includes from 5′→3′: (i) a first promoter for expressing a gene of interest in a cell (P1); (ii) a first Golden Gate site for cloning (GG1) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); (iii) optionally a counter selectable marker, for example, a toxin such as ccdB; (iv) a sixth Golden Gate site for cloning (GG6) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to its reverse complement overhang); and a third transcription termination site (T3).

The disclosed vectors also include one or more donor vectors that are cell-free expression vectors for expressing a gene of interest. By example, the embodiment of FIG. 1 has three donor vectors that are cell-free expression vectors, indicated as pDonor1, pDonor3, and pDonor5, which express Gene1, Gene2, and Gene3, respectively.

By example, pDonor1 comprises from 5′→3′: (i) a promoter for cell-free RNA synthesis (e.g., a promoter for T7 RNA polymerase); (ii) a first Golden Gate site for cloning (GG1) (i.e. a recognition site for a TypeIIS restriction enzyme such as a BsaI site or a BbsI site or an AarI site optionally oriented so as to cleave upstream (5′) of its recognition site and provide an overhang that hybridizes to the overhang of GG1 in pMTL8225-P-GG); (iii) optionally a first gene of interest (Gene1); and (iv) a second Golden Gate site for cloning (GG2) (i.e. a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to its reverse complement overhang); where the optional Gene1 is inserted between GG1 and GG2.

The disclosed vectors also include one or more donor vectors that comprise a donor promoter for use in expressing a gene of interest in a cell. The one or more donor vectors that comprise a donor promoter for use in expressing a gene of interest in a cell together may comprise a library of promoters of differing strengths. By example, the embodiment of FIG. 1 has two donor vectors that comprise a donor promoter for expression in a cell of a gene of interest, indicated as pDonor2 and pDonor 4.

By example, pDonor2 comprises from 5′→3′: (i) a second Golden Gate site for cloning (GG2) (i.e. a recognition site for a TypeIIS restriction enzyme such as a BsaI site or a BbsI site or an AarI site oriented so as to cleave outside of its recognition site and provide an overhang (e.g., a 5′ overhang) that hybridizes to the overhang of GG2 in pDonor1; (ii) a first transcription termination site (T1); (iii) a second promoter for expressing a gene of interest in a cell (P2) and a third Golden Gate site for cloning (GG3) (i.e. a recognition site for a TypeIIS restriction enzyme such as a BsaI site or a BbsI site or an AarI site oriented so as to cleave outside of its recognition site and provide an overhang (e.g., a 5′ overhang).

By example, pDonor3 comprises from 5′→3′: (i) a promoter for cell-free RNA synthesis (e.g., a promoter for T7 RNA polymerase); (ii) a third Golden Gate site for cloning (GG3) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to the overhang of GG3 in pDonor2); (iii) optionally a second gene of interest (Gene2); and (iv) a fourth Golden Gate site for cloning (GG4) (i.e. a recognition site for a TypeIIS restriction enzyme such as a BsaI site or a BbsI site or an AarI site optionally oriented so as to cleave downstream (3′) of its recognition site and provide an overhang (e.g., a 5′ overhang) that hybridizes to its reverse complement overhang); where the optional Gene2 is inserted between GG3 and GG4.

By example, pDonor4 comprises from 5′→3′: (i) a fourth Golden Gate site for cloning (GG4) (i.e. a recognition site for a TypeIIS restriction enzyme such as a BsaI site or a BbsI site or an AarI site oriented so as to cleave outside of its recognition site and provide an overhang (e.g., a 5′ overhang) that hybridizes to the overhang of GG4 in pDonor3; (ii) a second transcription termination site (T2); (iii) a third promoter for expressing a gene of interest in a cell (P3) and a fifth Golden Gate site for cloning (GG5) (i.e. a recognition site for a TypeIIS restriction enzyme such as a BsaI site or a BbsI site or an AarI site oriented so as to cleave outside of its recognition site and provide an overhang (e.g., a 5′ overhang) that hybridizes to its reverse complement overhang.

By example, pDonor5 comprises from 5′→3′: (i) a promoter for cell-free RNA synthesis (e.g., a promoter for T7 RNA polymerase); (ii) a fifth Golden Gate site for cloning (GG5) (i.e. a recognition site for a TypeIIS restriction enzyme such as a BsaI site or a BbsI site or an AarI site optionally oriented so as to cleave upstream (5′) of its recognition site and provide an overhang (e.g., a 5′ overhang) that hybridizes to the overhang of GG5 in pDonor4); (iii) optionally a third gene of interest (Gene3); and (iv) a sixth Golden Gate site for cloning (GG6) (i.e. a recognition site for a TypeIIS restriction enzyme such as a BsaI site or a BbsI site or an AarI site optionally oriented so as to cleave downstream (3′) of its recognition site and provide an overhang (e.g., a 5′ overhang) to the overhang of GG1 in pMTL8225-P-GG); where the optional Gene3 is inserted between GG5 and GG6.

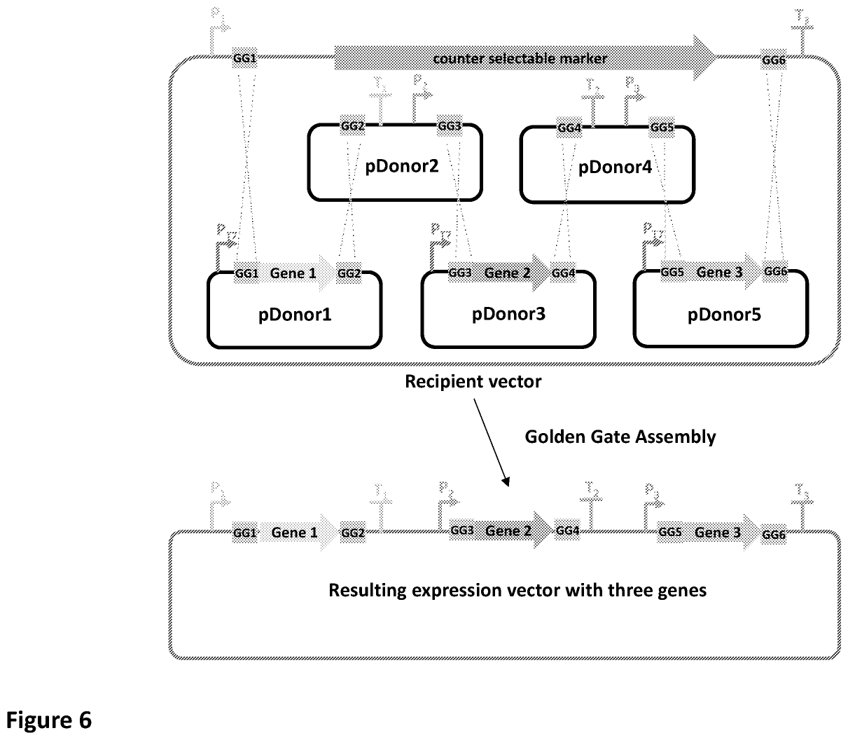

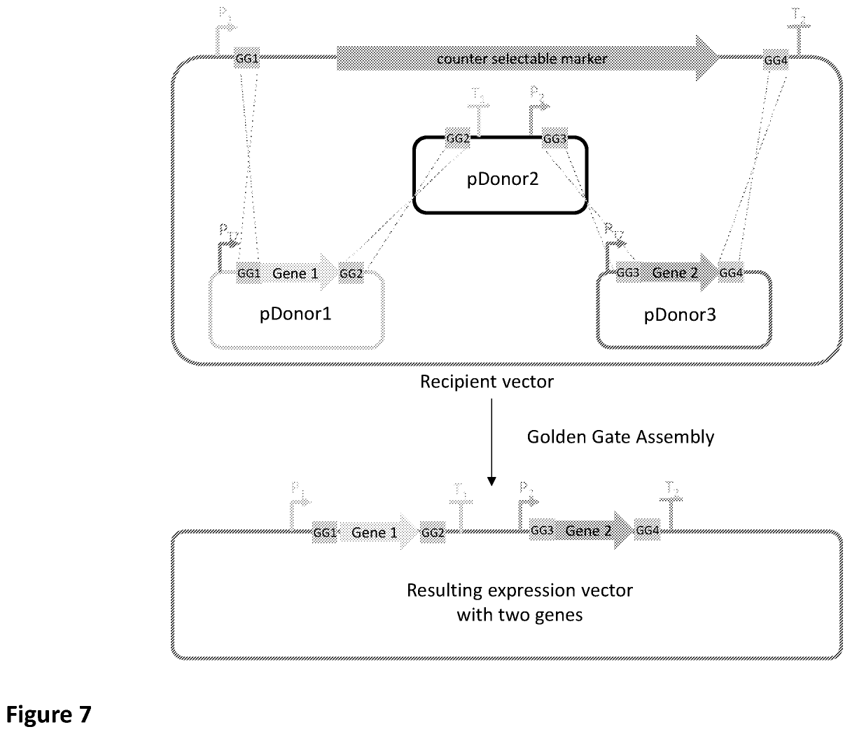

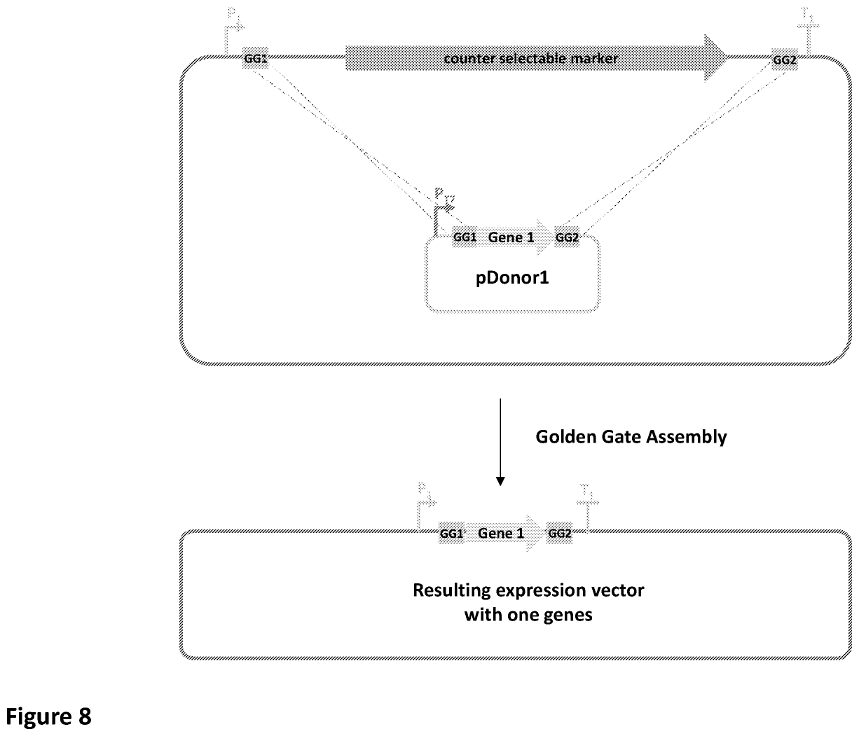

Designed as such, the vectors of the disclosed system can be utilized to express genes of interest in a cell free system, where one or more of pDonor1, pDonor2, and pDonor5, can be utilized to express one or more of Gene1, Gene2, and Gene3, respectively in a cell-free system. The inventors have expanded the system and built additional vectors that allow assembly of less than 3 genes. For example, for a two or one gene pathway, suitable donor and recipient vectors that enable assembly of 2 or 1 genes into the recipient vector can be utilized. (See FIGS. 6-8).

Further, the vectors of the disclosed systems can be utilized to provide a library of promoters having different strengths that can be used to express genes of interest in a cell, where pDonor2 and pDonor4 represent vectors that can provide promoters having different strengths that can be used to express genes of interest in a cell. Furthermore, designed as such through the use of Golden Gate sites, multiple promoters and genes can be inserted into a backbone expression vector, such as pMTL8225-P-GG, to provide a single vector for expressing multiple different genes using multiple different promoters.

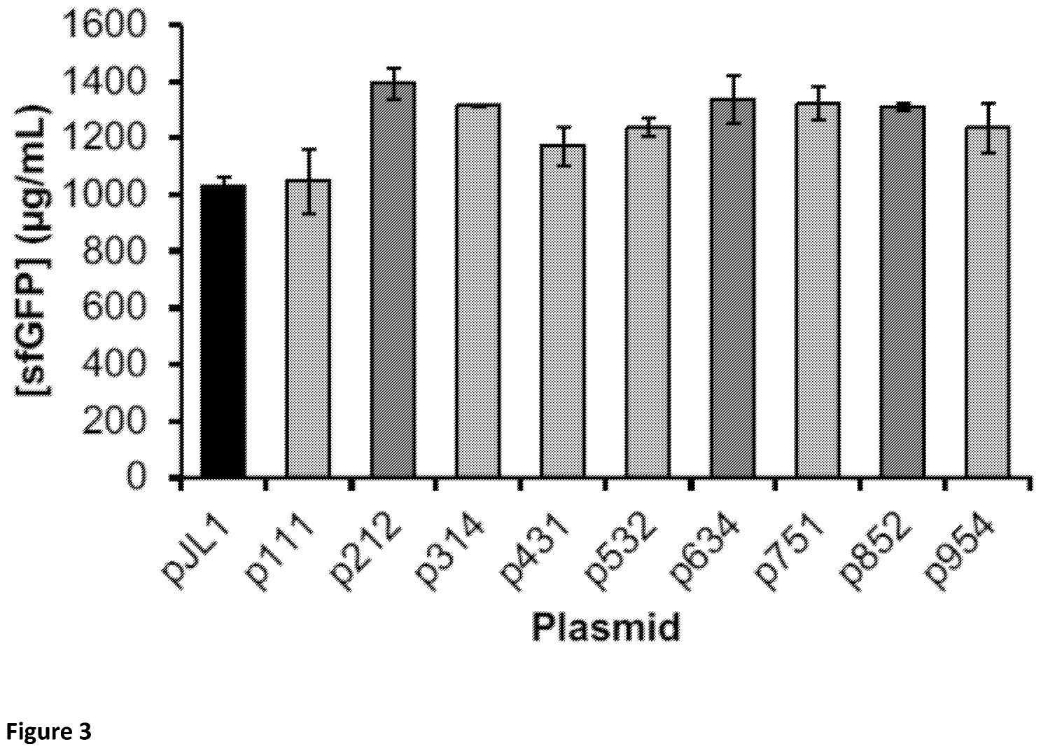

FIG. 2 illustrates various options for modifying cell-free expression vectors to include a Golden Gate site. As illustrated, the original expression vector comprising a T7 promoter was modified to remove an upstream BsaI site (positions 20-25), which creates a ⅕ overhang upstream overhang. BsaI sites or BbsI sites were created downstream of the ribosome binding site (RBS), which either left the RBS sequence or changed the RBS sequence. Cell-free expression vectors comprising modifications as such exhibited expression levels in cell-free systems that were at least as good as the unmodified vector. (See FIG. 3).

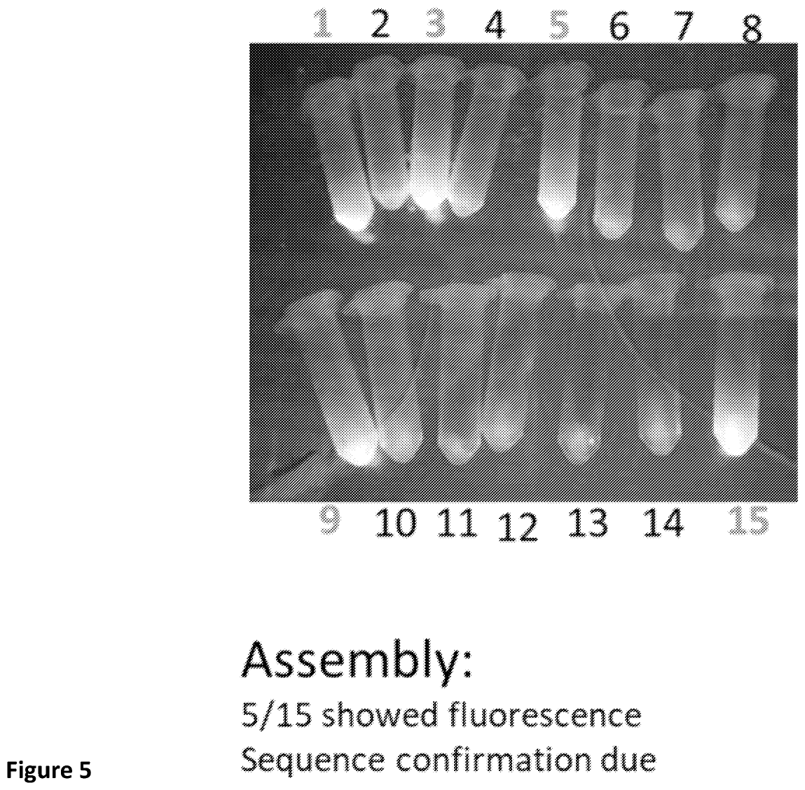

Using the donor plasmids, various components of a pathway for sfGFP expression were assembled into a backbone vector for expression in a cell. (See FIG. 4). Five of fifteen cells that were transformed with the assembled expression vector exhibited fluorescence. (See FIG. 5).

### Example 2—Modular Cell-Free Expression Plasmids to Accelerate Biological Design in Cells

Reference is made to the draft manuscript entitled “Modular cell-free expression plasmids to accelerate biological design in cells,” authored by Karim, A. S. et al., the content of which is provided below.

Abstract

Industrial biotechnology aims to produce high-value products from renewable resources. This can be challenging because model microorganisms-organisms that are easy-to-use like Escherichia coli—often lack the machinery required to utilize desired feedstocks like lignocellulosic biomass or syngas. Non-model organisms, such as Clostridium, are industrially proven and have desired the metabolic features but have several hurdles to mainstream use. Namely, these species grow more slowly than conventional laboratory microbes and genetic tools for engineering them are far less prevalent. To address these hurdles for accelerating cellular design, cell-free synthetic biology has emerged as an approach for characterizing non-model organisms and rapidly testing metabolic pathways in vitro. Unfortunately, cell-free systems can require specialized DNA architectures with minimal regulation that are not compatible with cellular expression. In this work, we develop a modular vector system that allows for T7 expression of desired enzymes for cell-free expression and direct Golden Gate assembly into Clostridium expression vectors. Utilizing the Joint Genome Institute's DNA Synthesis Community Science Program, we designed and synthesized these plasmids and genes required for our projects allowing us to shuttle DNA easily between our in vitro and in vivo experiments. We next validated that these vectors were sufficient for cell-free expression of functional enzymes, performing on par with the previous state-of-the-art. Lastly, we demonstrated automated six-part DNA assemblies for C. autoethanogenum expression with efficiencies ranging from 68-90%. We anticipate this system of plasmids will enable a framework for facile testing of biosynthetic pathways in vitro and in vivo by shortening development cycles.

Introduction

Industrial biotechnology often seeks to produce chemical products from inexpensive and prevalent feedstocks, such as lignocellulosic biomass and syngas.¹⁻³ While most synthetic biologists work with model organisms like Escherichia coli and Saccharomyces cerevisiae due to their ease-of-use, these organisms can be limited by accessible feedstocks, products, and stable operating environments in which to work. For example, these organisms do not naturally possess the metabolic pathways required to access the carbon in syngas; rather, researchers turn to diverse genera of non-model organisms and pathways capable of these unique biochemical transformations.⁴ One such genus is Clostridium, which includes the cellulolytic C. thermocellum as well as the gas-fermenting, acetogenic C. autoethanogenum.⁵⁻⁷ Despite their utility for biotechnology and commercial deployment, these species grow more slowly than conventional laboratory microbes, are obligate anaerobes, and genetic tools for engineering them are still developing and far less prevalent.

Developments in cell-free synthetic biology have sought to characterize non-model organisms⁸⁻¹⁰ and rapidly test metabolic pathways in vitro.¹¹⁻¹⁴ By using cell-free gene expression (CFE)¹⁵ to produce enzymes directly in vitro, metabolic pathways can be tested without the need to re-engineer organisms or construct new DNA elements between each engineering cycle.^{13,16-20} This approach benefits from the ability to test more enzyme variants, the ability to precisely tune reaction conditions and enzyme concentrations, and shorter engineering cycles to down-select promising candidate pathways for in vivo biochemical production.²¹ While cell-free pathway prototyping is carried out in a mix-and-match fashion,¹³ cellular expression requires assembly into operons. Additionally the specialized plasmids for Clostridium expression and those for CFE are not inherently compatible, for example require different promoters (cell-free expression typically relies on the orthogonal T7 system) and additional elements such as Gram-positive replication origin, specific antibiotics cassettes and a low GC content.²² This means Clostridium optimized DNA for successful pathway designs identified in vitro must be separately synthesized and cloned prior to transformation in Clostridium, adding several weeks of effort and considerable costs. Streamlining this process would increase the ability to engineer non-model organisms for metabolic engineering applications.

In this work, we present a modular plasmid system on the basis of standard cell-free vector pJL1^{10,19,23,24} and universal Clostridium shuttle vector system pMTL80000²² to rapidly bridge cell-free prototyping efforts and strain engineering in C. autoethanogenum and reduce the overall engineering cycle time. Engineering compatible expression plasmids requires fine tuning to minimize impacts on the genetic context of open reading frames, particularly around the critically important ribosome binding site.^{13, 19} First, we designed several plasmid architectures that resemble our top-performing cell-free expression vector, pJL1 (Addgene #69496), and would enable flexible arrangement of genes and promoters (Golden Gate assembly²⁵ compatibility) for expression in C. autoethanogenum. Next, we validated that these new vectors are sufficient for CFE of biosynthetic enzymes that are functionally active. Then, we demonstrated DNA assembly efficiency ranging from 68-90% when assembling up to six parts for C. autoethanogenum expression. We finally showed automation of the whole workflow on two different automation systems. This modular ‘Cell-free to Clostridium’ vector system along with high-throughput and automatable workflows will accelerate strain development efforts for C. autoethanogenum or other Clostridium species. Principles learned here, or perhaps the vectors themselves, could also accelerate biological design in other non-model organisms, by decreasing delays in the transition between cell-free prototyping and cellular validation.

Materials and Methods

Strains and Plasmids. For generation of the ‘Cell-free to Clostridium’ vector system and cloning, E coil strain TOPtO (Invitrogen) was used. First, the counter-selectable marker ccdB (flanked with BsaI recognition sites) was cloned into pMTL82251 and pMTL83151²² to generate the recipient Clostridium expression vectors (pCExpress). The construction of vectors pD2 and pD4 involved TOPO (Invitrogen) cloning of terminator and promoter parts (flanked with BsaI recognition sites) amplified or synthesized by JGI into the plasmid, pCR-blunt (Invitrogen). The ‘Cell-free to Clostridium’ vectors were derived from pJL1 plasmid (Addgene #69496), modified in the T7 promoter region to contain a BsaI recognition site between the RBS and START codon in three variations to generate pD1, pD3, and pD5. All recipient and donor vectors were verified by DNA sequencing.

DNA codon-optimized genes for C. autoethanogenum were generated using LanzaTech's in-house codon optimization software. E. coli adapted sequences were generated using codon optimization tools from Twist Biosciences (California, USA). Genes of interest were provided by JGI in the ‘Cell-free to Clostridium’ vectors pD1, pD3, and pD5. All vector DNA sequences used in this study are listed in Table 1, and all DNA parts are listed in Table 2. The 58 modular vectors containing parts from Table 2 are listed in Table 3. The biosynthetic genes used in cell-free assays are listed in Table 4, and those used in GG assembly are listed in Table 5.

Cell-free assays. All cell extracts for CFE were prepared with Escherichia coil BL21 Star(DE3) (NEB).²¹ These cells were grown, harvested, lysed, and prepared using previously described methods.^{19,26} CFE reactions were performed to express each enzyme individually using a modified PANOx-SP system at the 15- or 30-μL volumes in 2-mL Eppendorf tubes as described in previous publications.^{27,28} Protein measurements were taken after 20 h. Active super-folder GFP (sfGFP) protein yields were quantified by measuring fluorescence. To do this, two microliters of the total CFE reaction were added in the middle of the flat bottom of 96-well half area black plates (Costar 3694; Corning Incorporated, Corning, N.Y.). sfGFP was excited at 485 nm while measuring emission at 528 nm with a 510 nm cutoff filter. The fluorescence of sfGFP was converted to concentration (μg/mL) according to a standard curve.²⁹ All other proteins were measured using CFE reactions with radioactive ¹⁴C-Leucine (10 μM) supplemented for incorporation during protein production. We used trichloroacetic acid (TCA) to precipitate radioactive protein samples. Radioactive counts from TCA-precipitated samples was measured by liquid scintillation to then quantify soluble and total yields of each protein produced as previously reported (MicroBeta2; PerkinElmer).^{27,30}

The cell-free activity assays were run at 15-μL volumes in 1.5-mL Eppendorf tubes. All enzyme-enriched lysates (via CFE reaction) were added at 0.4 μM concentration of enriched enzyme as determined by C¹⁴ measurements with the balance being ‘blank CFE’ reaction (no DNA added) up to 50% of the total reaction volume. Small molecules were added to achieve final concentrations of 120 mM glucose, 3 mM NAD⁺, 5 mM CoA, 100 mM BisTris pH 7, 8 mM magnesium acetate salts, 0.1 U/μL catalase, in the reaction. Reactions were run for 20 h, quenched with 5% TCA, and measured via HPLC as described previously.^{13,19}

Golden Gate assembly using manual workflow. Two- to six-part DNA assemblies were performed using GeneArt Type IIs (BsaI) assembly kit (Invitrogen, CA). Specifically, 75 ng of recipient vector was used. Other parts (pD1, pD2, pD3, pD4, and pD5) were added in 1:1 molar ratio with respect to the recipient vector along with the GeneArt Type IIs enzyme mix. The reaction was then incubated in a thermocycler (37° C. for 1 min, 16° C. for 1 min, cycled 30×, followed by cooling at 4° C.). Afterwards, the assembly mixture was transformed into E. coli Top 10 chemically competent cells (ThermoFisher Scientific, CA), and plated onto LB agar containing appropriate antibiotics. Resulting colonies were screened via PCR for presence of the parts cloned, followed by sequence confirmation via NGS.

Golden Gate assembly using automated workflow. Two automated assembly workflows were developed, either using the liquid handling robot Hamilton STARLet or the Labcyte Echo 525.³¹ The assembly reactions were carried out with the final concentration for each individual DNA part was 2 nM.²⁵ The assembly reaction volume for Hamilton STARLet was 20 μL prepared as follows: 2 μL of each DNA part (10 nM), 10 μL GeneArt Type IIs Assembly Kit BsaI (Invitrogen A15917), and deionized water to a total of 20 μL. When using the Labcyte Echo 525, the reaction volume was downsized to a final volume of 2 μL. All DNA samples were quantified by absorbance at 280 nm, employing a NanoDrop 2000 spectrophotometer (Thermo Fisher Scientific). Reactions were incubated in an INHECO heat block using the following parameters: 37° C. for 2 h, 50° C. for 5 min, 80° C. for 10 min, then stored at −20° C. until transformation. Transformations were also performed using the INHECO blocks: 2 μL of each reaction mix was added to 20 μL of Invitrogen One Shot Top10 chemically competent cells (C404003) and incubated for 20 min at 4° C. Cells were then heat-shocked at 60° C. for 45 sec, then recovered for 2 min at 4° C. Afterwards, 180 μL of Super Optimal Broth (Invitrogen 15544034) with catabolite repression (SOC) media was added to cell mixtures, and cells were recovered at 37° C. for 2 h. Finally, 7 μL of each transformation reaction consisting of undiluted culture volume was plated on Lennox Lysogeny Broth (LB)+agar plates containing the appropriate antibiotic and incubated overnight at 37° C. We randomly chose two colonies to sequence throughout the assembled regions. NGS sequencing confirmed that more than 90% of the clones screened showed complete assemblies.

Results and Discussion

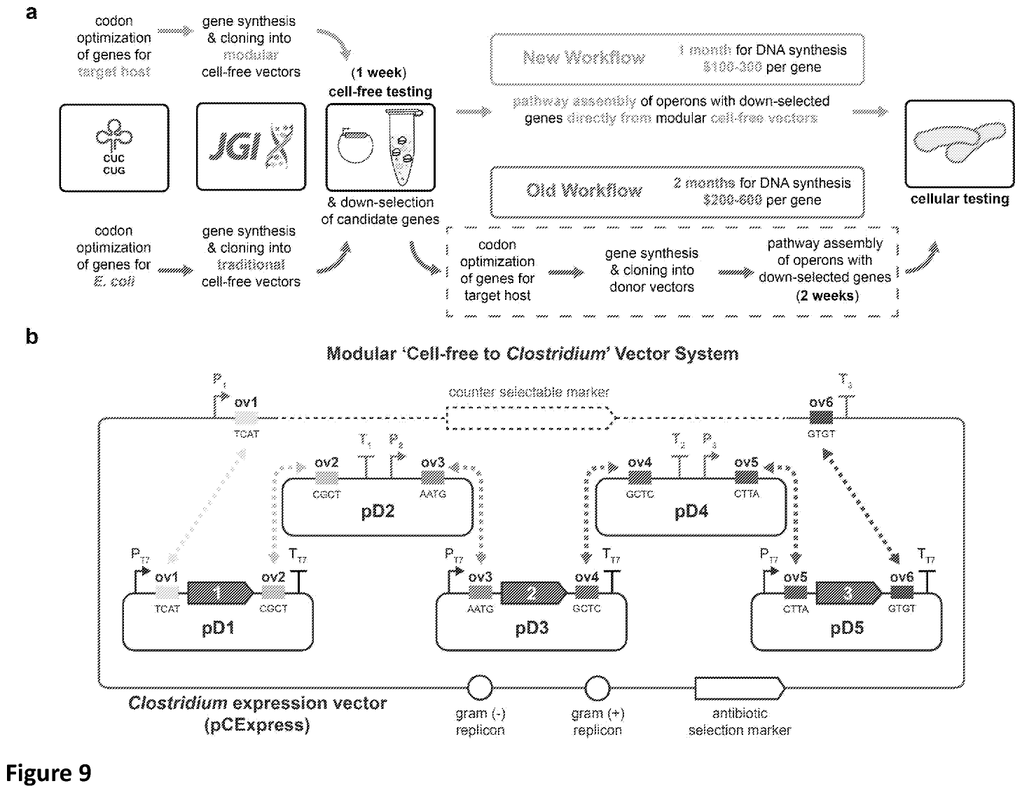

Design of a modular ‘cell-free to Clostridium’ vector system. Our goal was to develop a DNA vector system that would enable easy exchange of DNA between cell-free plasmids and cellular plasmids. This cell-free to Clostridium framework would minimize repetitive DNA synthesis and subcloning and enable re-use of DNA, allowing for facile and rapid testing of biosynthetic pathways in vitro and in vivo. Traditional approaches required optimization and synthesis of candidate genes for cell-free expression and cell-free testing followed by separate optimization and synthesis of genes for Clostridium expression (FIG. 9A), taking significant time and slowing down research efforts. The workflow presented here allows for a single round of codon optimization and synthesis for Clostridium that can be used for both cell-free testing and pathway assembly in Clostridium (FIG. 9A), reducing the time and cost of research and development in these complex organisms. Specifically, there is a 50% reduction in DNA synthesis time and cost as well as only one round of cloning into cell-free/donor vectors prior to in vivo vector assembly. To achieve this goal, CFE expression plasmids were modified via addition of Golden Gate (GG) sites so that these can be used directly for multiple DNA parts assembly directly into Clostridium expression vectors (FIG. 9B). With such a system in place, genes could be ordered once with Clostridium codon-adapted sequences in these plasmids, prototyped in cell-free reactions using these plasmids, and then the best performing gene variants could be assembled from the same plasmids into in vivo expression plasmids via one-step GG assembly.

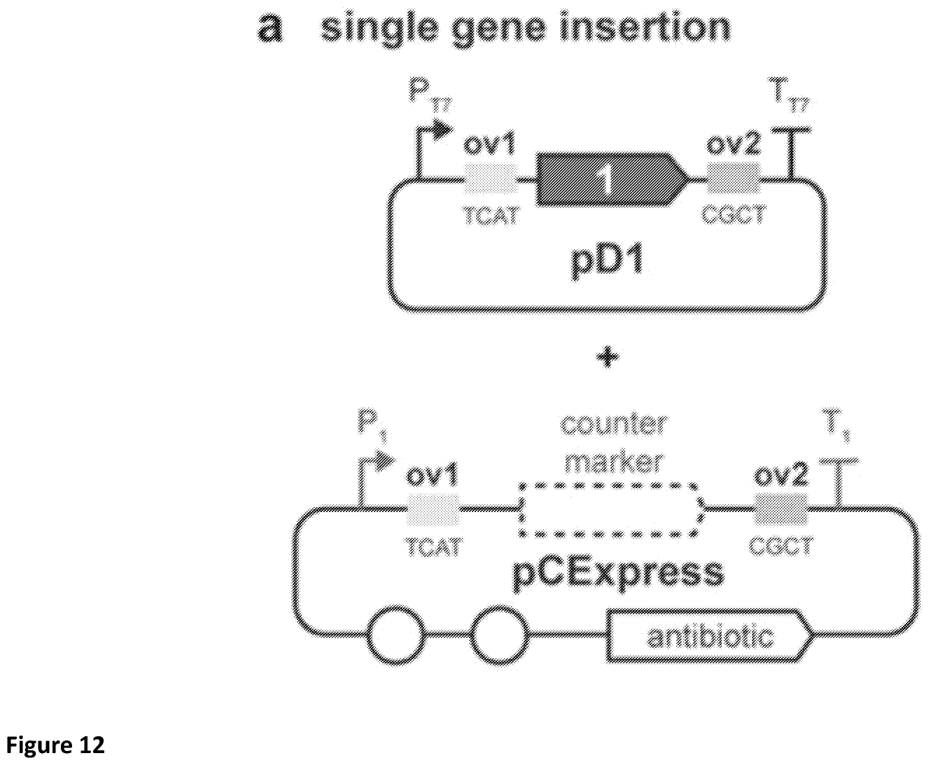

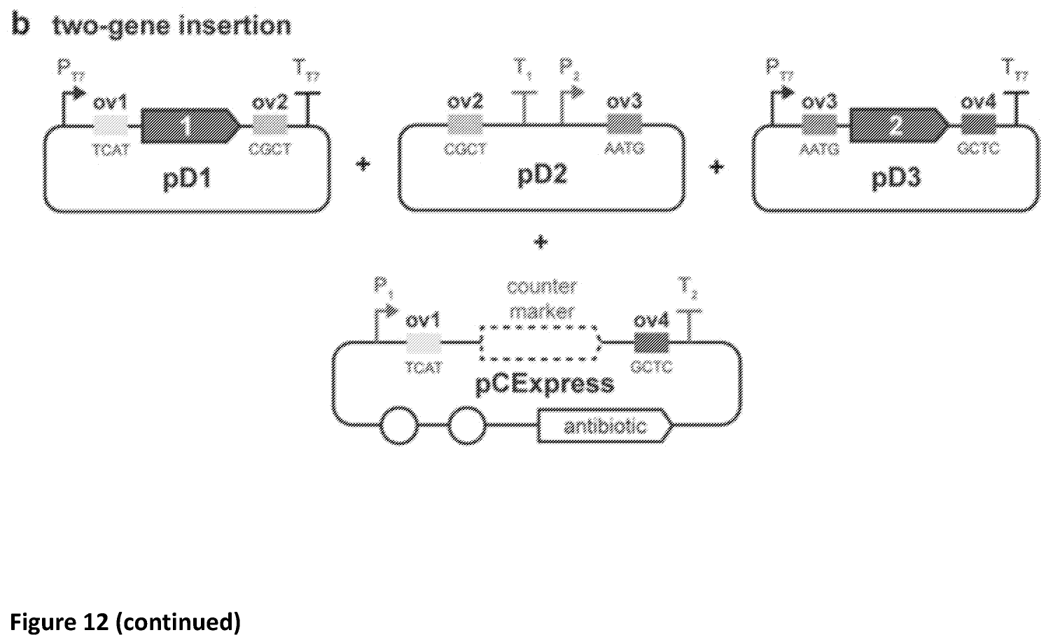

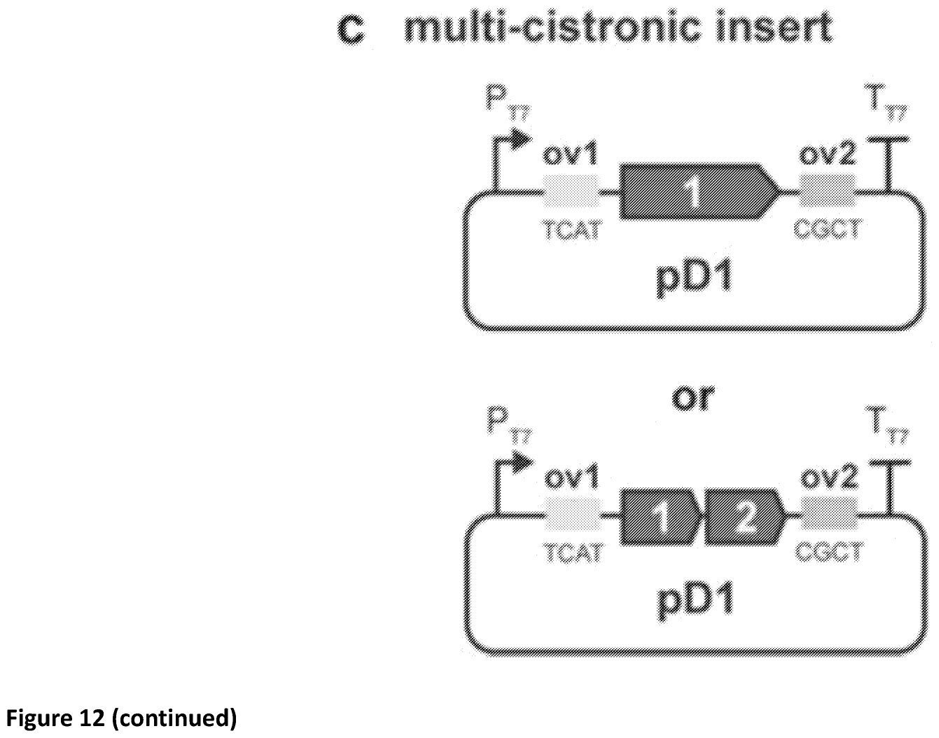

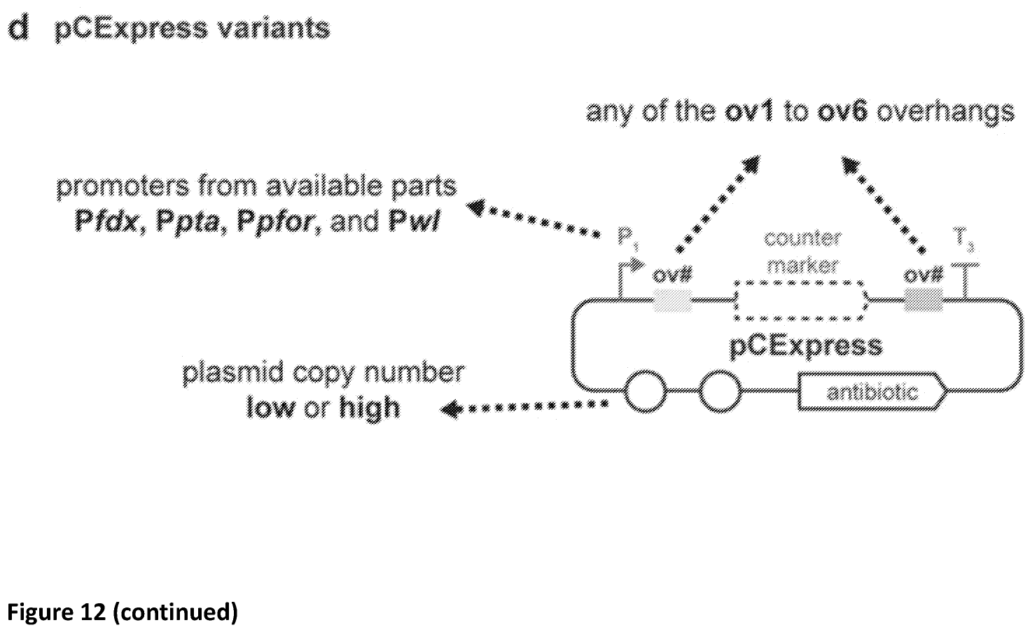

As a starting point, we designed six total vectors. Three vectors, pD1, pD3, and pD5, were constructed by adding GG sites (BsaI recognition sites) within the T7 promoter region of the pJL1 vector (Addgene #69496), a standard CFE expression plasmid. These vectors were designed to serve as gene donor vectors for assembly in a new recipient vector based on pMTL80000 universal Clostridium expression vectors²² with the addition of two GG sites flanking a ccdB survival gene along with Clostridium promoter and terminator flanking the GG sites. We also constructed pD2 and pD4 to serve as promoter-terminator donor vectors. This system of six vectors (5 donor vectors and 1 recipient vector) would allow for in vitro expression of genes using pD1, pD3, and pD5, followed by one-step assembly of up to six DNA parts (inserts supplied by pD1, pD2, pD3, pD4, and pD5) directly into our Clostridium expression vector. We note that different combinations of these vectors can be used to assemble one-gene insertions (FIG. 12A) or two-gene insertions (FIG. 12B) when fewer genes are desired. It is also possible to combine more than three genes in an expression operon by using multi-cistronic donor vectors (FIG. 12C).

Utilizing the Joint Genome Institutes' (JGI's) gene synthesis program, the GG system was expanded to create recipient and donor vectors with varying promoters, such as P_{fdx}¹⁷, Pₚₜₐ¹⁸, P_{pfor}¹⁹, and P_{w1}¹⁸. Additionally GG sites were varied in recipient vectors to allow assembly of anywhere between two to six parts with varying promoters, resulting in a total of 58 modular vectors (FIG. 12D; Table 3). The variety of assembly options using different DNA parts (Table 2) increases the versatility of this vector system.

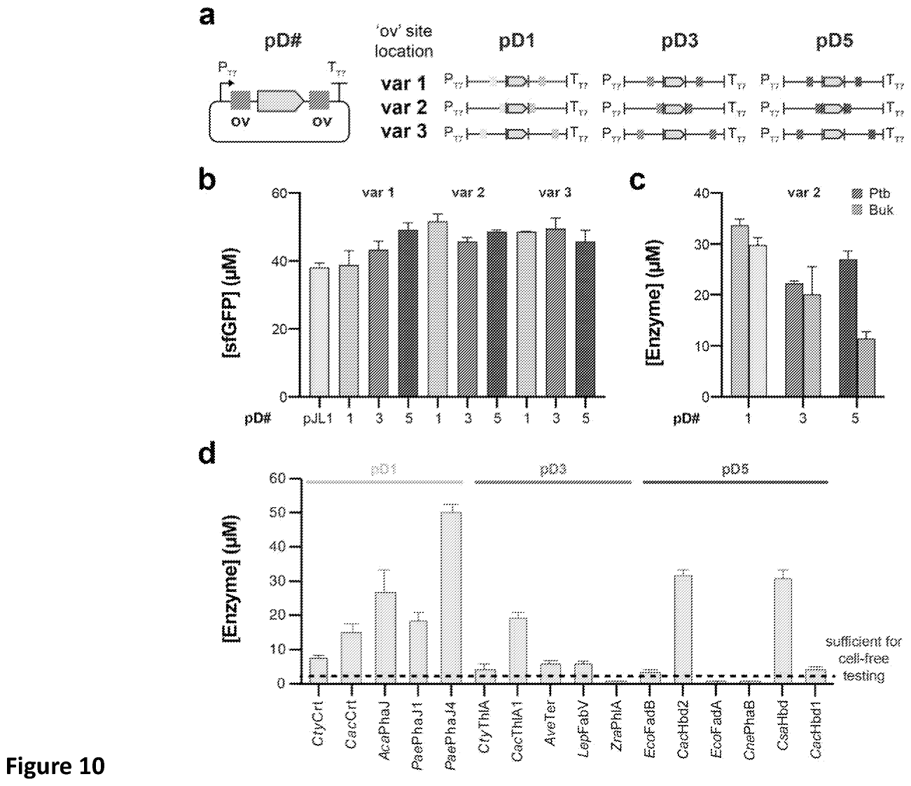

Evaluation of the vector system for CFE. The highest-yielding cell-free systems take advantage of T7 RNA polymerization and are substantially affected by changes in plasmid architecture.^{32,33} For example, our previous work using the pJL1 vector that leverages T7 RNA polymerase to make mRNA achieved protein yields of ˜2.7 g/L super-folder green fluorescent protein (sfGFP).³¹ In order to adapt our robust pJL1 vector for GG compatibility, we chose to test the insertion of three BsaI site designs in pJL1 (FIG. 10A; Table 2). Specifically, GG sites with sequences TCAT, AATG, or CTTA were introduced between the ribosome binding site (RBS) and start codon, which increased the spacer length between these two elements by 1 to 3 nucleotides. This created three distinct donor vectors each with three possible BsaI cut sites. We evaluated each of these nine designs in a cell-free gene expression reaction based on the PANOx-SP system to produce sfGFP to first assess the impact of GG sites on protein expression. After 20 h, cell-free reactions produced comparable or slightly greater concentrations of sfGFP than the unaltered pJL1 plasmid (FIG. 10B). For the donor vectors to be compatible we had to choose the same variant for each of the three vectors. Thus, we chose variant 2 as the highest-performing set. Variant 2 GG vectors were validated further with expression of the enzymes phosphotransbutyrylase (Ptb) and butyrate kinase (Buk) from butyric acid metabolism in C. acetobutylicum ATCC 824 (FIG. 10C). This experiment highlights the importance of genetic context for expression of different proteins, yielding variable amount of protein for Ptb and Buk despite nearly identical expression of sfGFP.

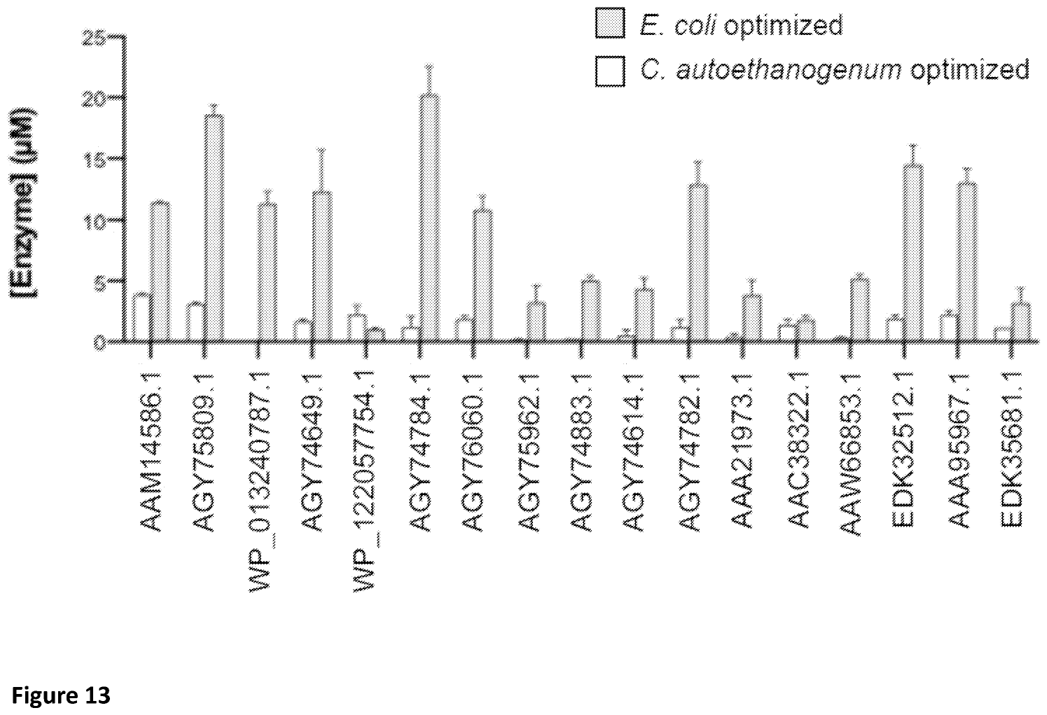

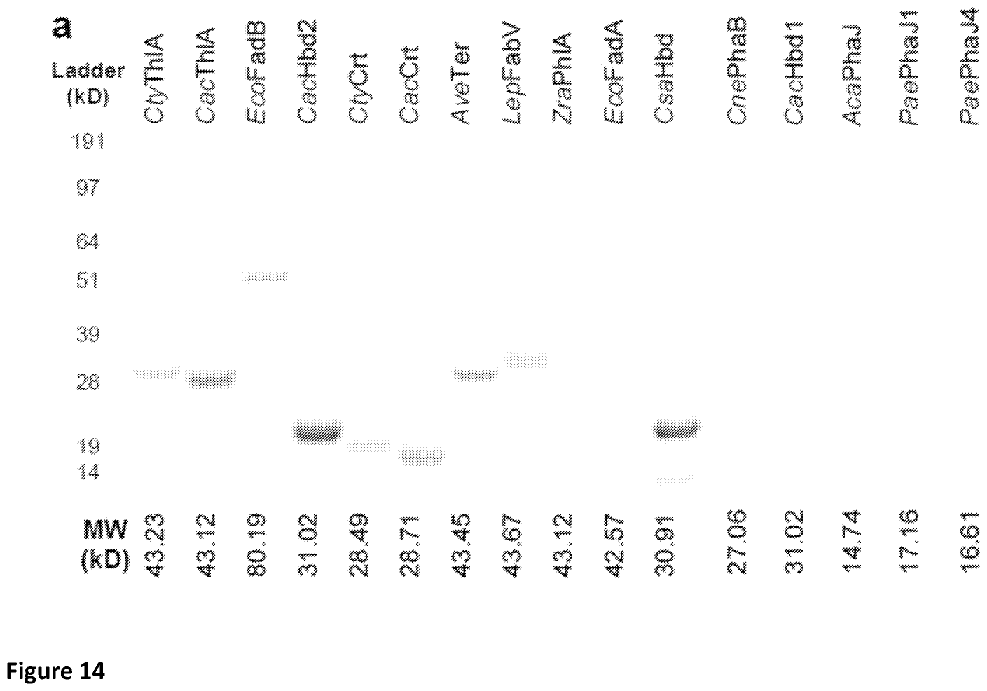

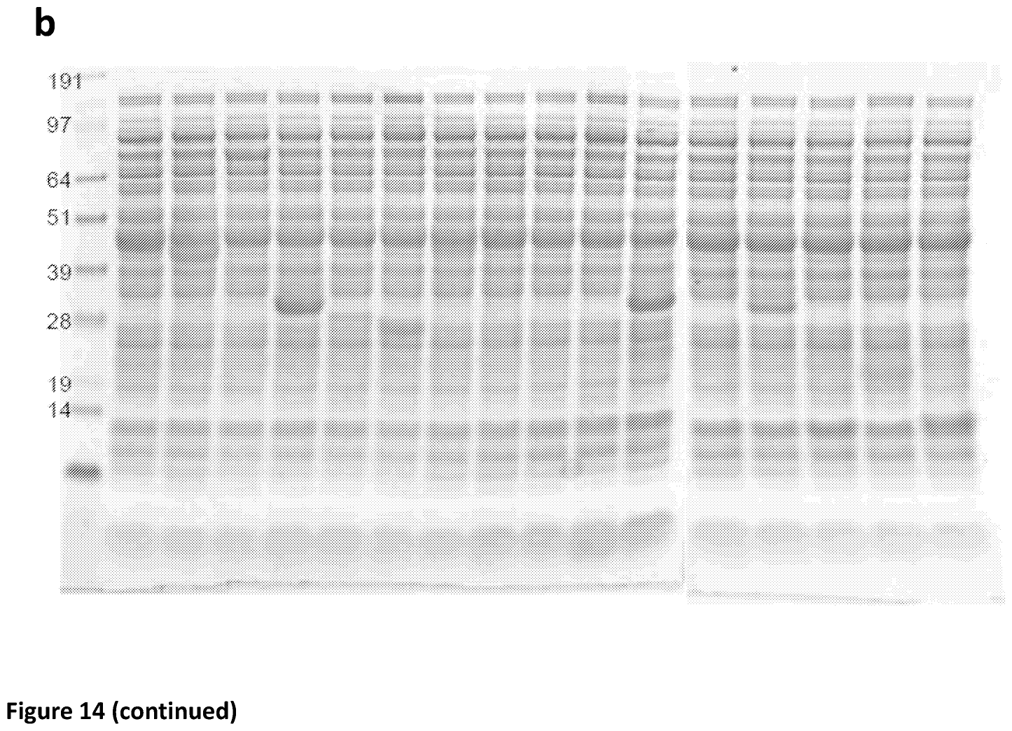

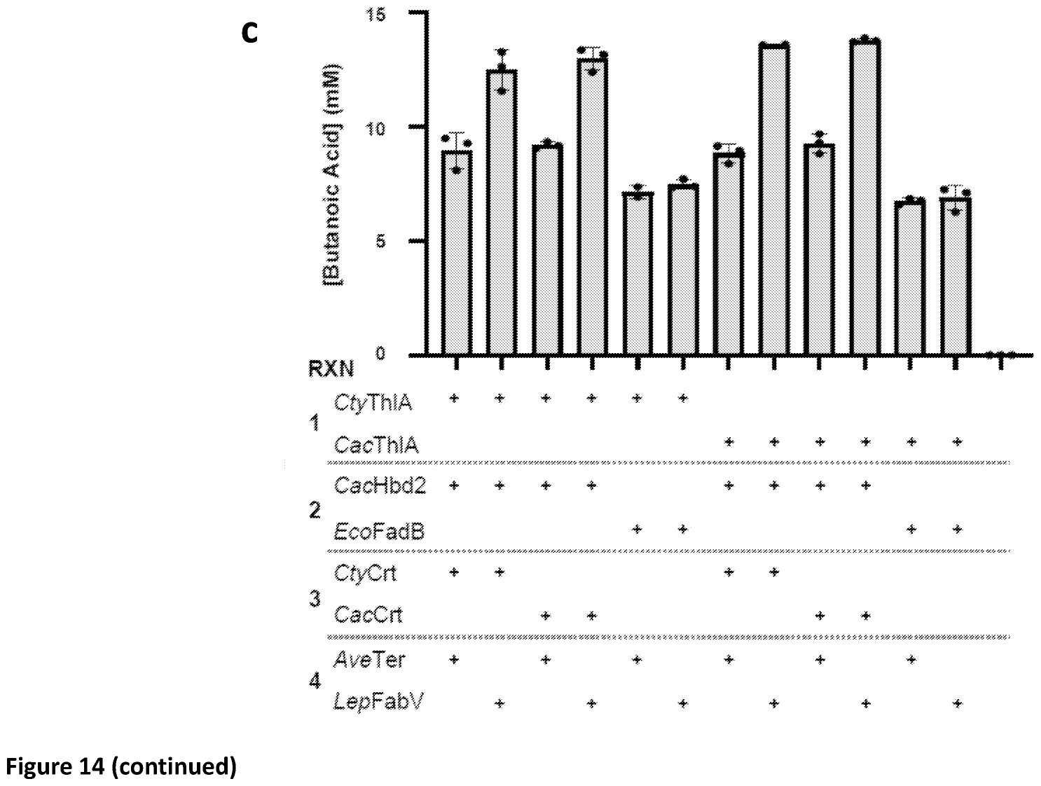

We next evaluated whether C. autoethanogenum-optimized genes would be sufficiently expressed in our established cell-free assays. To test this, we constructed a panel of 16 Clostridium-optimized biosynthesis genes (Table 4) related to acid/alcohol fermentation from a variety of organisms in pD1, pD3, and pD5. Several of these genes were used in a previous study for the production of butanol from acetyl-CoA, while the rest were identified through sequence similarity to these genes. After a 20-h CFE reaction we observed a range of expression all significantly lower (10-fold) than what we saw for E. coli-codon-optimized sfCFP, Ptb, or Buk (FIG. 10D). However, expressing full-length enzymes at concentrations greater than 1 μM provides at least 0.1 μM enzyme after dilution upon pathway assembly in vitro which we have found to be sufficient for prototyping.¹³ Thus, we proceeded to test whether C. autoethanogenum-optimized sequences were sufficient for prototyping biosynthetic pathways. First, we ran the 16 enzymes on a protein gel via SDS-PAGE followed by ¹⁴C autoradiography to confirm that indeed those enzymes that expressed, expressed at full-length (FIG. 14A, 14B). Decreasing the reaction temperature to slow down protein translation and folding and altering the DNA template sequence (e.g., RBS, coding sequence) can be pursued when important homologs are not expressed in soluble form. Then, we used these enzymes to construct biosynthetic pathways to produce butanoic acid to test the activity of these enzymes. This pathway runs from glucose to acetyl-CoA using native metabolism present in E. coli crude lysates followed by 4 enzymatic steps to produce butanoic acid. Mixing 12 combinations of the enzymes from FIG. 2D with glucose and cofactors, each combination produced >7 mM butanoic acid (FIG. 14C). These enzymes can be studied further using more detailed activity studies assays via purification and defined substrates. For enzymes that are not expressed well, separately expressing the E. coli-codon-optimized versions can improve enzyme expression (FIG. 13; Table 4). Although the soluble protein yields are generally lower using C. autoethanogenum (31% GC content) codon-optimized sequences in E. coli-based (50% GC content) cell extract, our data suggests that C. autoethanogenum optimized enzyme sequences are active in E. coli crude lysates. With GG-compatible vectors at hand we next sought construct in vivo expression plasmids.

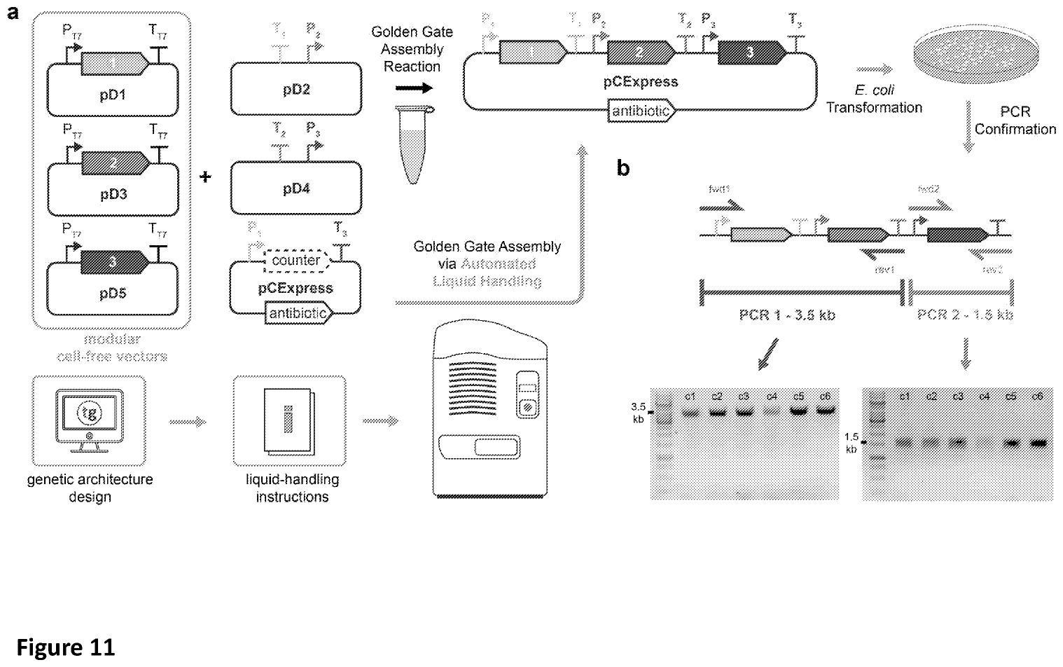

Six-part DNA assembly from CFE vectors into Clostridium expression plasmid. Once the pD vectors were successfully validated in CFE, these modified vectors were then used for testing the efficiency of multiple-part assembly directly into a Clostridium expression vector with a variety of biosynthetic genes. Specifically, we carried out a six-part GG assembly that contained: (i) a recipient vector based on pMTL8315 backbone containing a promoter (P1) and terminator (T3) flanking the two GG sites (pCExpress), (ii) pD2 and pD4, both containing terminator and promoter combinations (i.e., TT-P2 and T2-P3), and (iii) pD1, pD3, and pD5 containing gene 1, gene 2, and gene 3 (FIG. 11A; Table 1). The assembly mixture was transformed into our E. coli cloning strain and six colonies were picked and genotyped by PCR which indicated 90-100% of the picked colonies had plasmids with all six parts correctly assembled (FIG. 11B). These were confirmed via sequencing. The six-part assembly was validated using a different set of genes and promoter-terminator combinations for at least five additional designs (Table 5). In a total of 20 manual assemblies were carried out with efficiency ranging from 70-95%.

Assembled constructs can then be transformed into Clostridia to test for biosynthetic pathway activity. We previously showed that optimization of pathways in an E. coli cell-free system could inform cellular design in Clostridia.¹³ This work showed positive correlations between cell-free activity data and in vivo expression. Down-selecting a fraction of the >200 pathway combinations tested in vitro for constructing in Clostridia saved more than six months of research efforts. Combining this modular vector system with Clostridia-based cell-free expression¹⁰ should lead to more positive correlations and a streamlined research pipeline.

Workflow automation. Workflow automation can improve throughput and reliability. CFE reactions can routinely be performed using liquid-handling robotics.^{35,36} These reactions can be scaled down to 2 μL without significant changes in protein expression.³⁷ In addition, GG assembly for in vivo expression can also be automated. After demonstrating successful assembly of up to six DNA parts using a manual workflow, we then developed an automated workflow to increase our DNA assembly throughput (FIG. 11A). Due to the complexity of biological systems, it is often necessary to test a large number of enzyme homologs along with different promoters to obtain an optimal engineering solution. Indeed, testing just five homologs and three promoters for a three gene operon would yield 3,375 different permutations. However, this experimental throughput is difficult and laborious when using manual techniques and procedures. Automated, well-informed designs help to increase the number of designs that can be generated, the speed these designs can be generated, and it helps to narrow down the design space prioritizing the best candidates to be built and tested, saving lab resources.³⁸ In order to increase throughput, efficiency, and accuracy of our strain engineering pipeline, free up researchers from repetitive tasks, and increase results reproducibility, we validated a Golden Gate DNA assembly automated protocol on two automation systems. Both the design of constructs and the worklists to run the experiments were generated by J5 software.^{39,40} We assembled three- to six-part GG assemblies using both a Hamilton STARLet liquid-handling robot and a Labcyte Echo 525 acoustic liquid-handling robot with greater than 90% efficiencies.

### CONCLUSION

In this study, we describe a set of modular vectors for both cell-free gene expression and cloning into Clostridium expression plasmids. This framework allows facile testing of biosynthetic pathways in vitro and in vivo for shorter engineering cycles and enables an improved workflow between our in vitro team, our in vivo team, and JGI, without lengthy and costly re-synthesis and/or subcloning. The ‘Cell-free to Clostridium’ vector system is easy to use for Golden Gate assembly of up to six parts (three open reading frames with unique promoter and terminator sequences) at once with up to 90% efficiencies and feeds directly into the JGI's Community Science Program platform. For longer operons, genes can be sequentially located on each of the CFE vectors (pD1, pD3, and pD5). These vectors along with laboratory automation have already increased the speed and efficiency of our workflows and will continue to facilitate the ability to prototype biosynthetic pathways in vitro followed by in vivo cloning pipelines. Standardization of these vector systems allows for new simplified workflows. The pJL1 cell-free vector and variants thereof are routinely used in multiple bacterial cell-free systems (i.e., E. coli,¹⁹ Clostridium,¹⁰ Pseudomonas,²³ Streptomyces,^{24,41} Vibrio natriegens^{42,43}). In addition, the pMTL vector system has been demonstrated in several Clostridia species (i.e., autoethanogenum, ljungdahlii, acetobutylicum, beijerinckii, difficile, sporogeneses, perfringens, pasteurianum, tyrobutyricum) as well as other Gram-negative and Gram-positive model organisms such as E. coli and Bacillus.^{22,44} Taken together, the breadth of bacterial cell-free systems that can use the pJL1 vector and ubiquity of Golden Gate cloning suggests broad applicability of our plasmid vector system. Looking forward, we anticipate this system of vectors will allow researchers to integrate more in vitro prototyping practices into their existing workflows across multiple organisms to speed up metabolic engineering efforts.

### REFERENCES

1 Clomburg, J. M., Crumbley, A. M. & Gonzalez, R. Industrial biomanufacturing: The future of chemical production. Science 355, doi:10.1126/science.aag0804 (2017).
2 Liu, Z., Wang, K., Chen, Y., Tan, T. & Nielsen, J. Third-generation biorefineries as the means to produce fuels and chemicals from CO2. Nature Catalysis 3, 274-288, doi:10.1038/s41929-019-0421-5 (2020).
3 Kopke, M. & Simpson, S. D. Pollution to products: recycling of ‘above ground’ carbon by gas fermentation. Curr Opin Biotechnol 65, 180-189, doi:10.1016/j.copbio.2020.02.017 (2020).
4 Yan, Q. & Fong, S. S. Challenges and Advances for Genetic Engineering of Non-model Bacteria and Uses in Consolidated Bioprocessing. Front Microbiol 8, 2060, doi:10.3389/fmicb.2017.02060 (2017).
5 Tracy, B. P., Jones, S. W., Fast, A. G., Indurthi, D. C. & Papoutsakis, E. T. Clostridia: the importance of their exceptional substrate and metabolite diversity for biofuel and biorefinery applications. Curr Opin Biotechnol 23, 364-381, doi:10.1016/j.copbio.2011.10.008 (2012).
6 Lynd, L. R. et al. in Industrial Biotechnology: Microorganisms Vol. 1 (eds C. Wittmann & J. C. Liao) (Wiley-VCH Verlag GmbH & Co. KGaA, 2016).
7 Marcellin, E. et al. Low carbon fuels and commodity chemicals from waste gases—systematic approach to understand energy metabolism in a model acetogen. Green Chemistry 18, 3020-3028, doi:10.1039/c5gc02708j (2016).
8 Yim, S. S. et al. Multiplex transcriptional characterizations across diverse bacterial species using cell-free systems. Mol Syst Biol 15, e8875, doi:10.15252/msb.20198875 (2019).
9 Moore, S. J. et al. Rapid acquisition and model-based analysis of cell-free transcription-translation reactions from nonmodel bacteria. Proc Natl Acad Sci USA 115, E4340-E4349, doi:10.1073/pnas.1715806115 (2018).
10 Krüger, A. et al. Development of a clostridia-based cell-free system for prototyping genetic parts and metabolic pathways. Metab Eng, doi:10.1016/j.ymben.2020.06.004 (2020).
11 Moore, S. J., MacDonald, J. T. & Freemont, P. S. Cell-free synthetic biology for in vitro prototype engineering. Biochem Soc Trans 45, 785-791, doi:10.1042/BST20170011 (2017).
12 Jiang, L., Zhao, J., Lian, J. & Xu, Z. Cell-free protein synthesis enabled rapid prototyping for metabolic engineering and synthetic biology. Synth Syst Biotechnol 3, 90-96, doi:10.1016/j.synbio.2018.02.003 (2018).
13 Karim, A. S. et al. In vitro prototyping and rapid optimization of biosynthetic enzymes for cell design. Nat Chem Biol 16, 912-919, doi:10.1038/s41589-020-0559-0 (2020).
14 Liu, Z. et al. In Vitro Reconstitution and Optimization of the Entire Pathway to Convert Glucose into Fatty Acid. ACS Synth Biol 6, 701-709, doi:10.1021/acssynbio.6b00348 (2017).
15 Silverman, A. D., Karim, A. S. & Jewett, M. C. Cell-free gene expression: an expanded repertoire of applications. Nat Rev Genet 21, 151-170, doi:10.1038/s41576-019-0186-3 (2020).
16 Grubbe, W. S., Rasor, B. J., Krüger, A., Jewett, M. C. & Karim, A. S. Cell-free styrene biosynthesis at high titers. bioRxiv, 2020.2003.2005.979302, doi:10.1101/2020.03.05.979302 (2020).
17 Dudley, Q. M., Karim, A. S., Nash, C. J. & Jewett, M. C. Cell-free prototyping of limonene biosynthesis using cell-free protein synthesis. BioRxiv, 2020.2004.2023.057737, doi:10.1101/2020.04.23.057737 (2020).
18 Kightlinger, W. et al. A cell-free biosynthesis platform for modular construction of protein glycosylation pathways. Nat Commun 10, 5404, doi:10.1038/s41467-019-12024-9 (2019).
19 Karim, A. S. & Jewett, M. C. A cell-free framework for rapid biosynthetic pathway prototyping and enzyme discovery. Metab Eng 36, 116-126, doi:10.1016/j.ymben.2016.03.002 (2016).
20 Dudley, Q. M., Nash, C. J. & Jewett, M. C. Cell-free biosynthesis of limonene using enzyme-enriched Escherichia coli lysates. Synth Biol (Oxf) 4, ysz003, doi:10.1093/synbio/ysz003 (2019).
21 Karim, A. S. & Jewett, M. C. Cell-Free Synthetic Biology for Pathway Prototyping. Methods Enzymol 608, 31-57, doi:10.1016/bs.mie.2018.04.029 (2018).
22 Heap, J. T., Pennington, O. J., Cartman, S. T. & Minton, N. P. A modular system for Clostridium shuttle plasmids. J Microbiol Methods 78, 79-85, doi:10.1016/j.mimet.2009.05.004 (2009).
23 Wang, H., Li, J. & Jewett, M. C. Development of a Pseudomonas putida cell-free protein synthesis platform for rapid screening of gene regulatory elements. Synthetic Biology 3, doi:10.1093/synbio/ysy003 (2018).
24 Li, J., Wang, H., Kwon, Y. C. & Jewett, M. C. Establishing a high yielding streptomyces-based cell-free protein synthesis system. Biotechnol Bioeng 114, 1343-1353, doi:10.1002/bit.26253 (2017).
25 Engler, C., Kandzia, R. & Marillonnet, S. A one pot, one step, precision cloning method with high throughput capability. PLoS One 3, e3647, doi:10.1371/journal.pone.0003647 (2008).
26 Kwon, Y. C. & Jewett, M. C. High-throughput preparation methods of crude extract for robust cell-free protein synthesis. Sci Rep 5, 8663, doi:10.1038/srep08663 (2015).
27 Jewett, M. C. & Swartz, J. R. Mimicking the Escherichia coli cytoplasmic environment activates long-lived and efficient cell-free protein synthesis. Biotechnol Bioeng 86, 19-26, doi:10.1002/bit.20026 (2004).
28 Jewett, M. C. & Swartz, J. R. Substrate replenishment extends protein synthesis with an in vitro translation system designed to mimic the cytoplasm. Biotechnol Bioeng 87, 465-472, doi:10.1002/bit.20139 (2004).
29 Hong, S. H. et al. Cell-free protein synthesis from a release factor 1 deficient Escherichia coli activates efficient and multiple site-specific nonstandard amino acid incorporation. ACS Synth Biol 3, 398-409, doi:10.1021/sb400140t (2014).
30 Jewett, M. C., Calhoun, K. A., Voloshin, A., Wuu, J. J. & Swartz, J. R. An integrated cell-free metabolic platform for protein production and synthetic biology. Mol Syst Biol 4, 220, doi:10.1038/msb.2008.57 (2008).
31 Walsh, D. I., 3rd et al. Standardizing Automated DNA Assembly: Best Practices, Metrics, and Protocols Using Robots. SLAS Technol 24, 282-290, doi:10.1177/2472630318825335 (2019).
32 Shin, J. & Noireaux, V. Efficient cell-free expression with the endogenous E. Coli RNA polymerase and sigma factor 70. J Biol Eng 4, 8, doi:10.1186/1754-1611-4-8 (2010).
33 Yeung, E. et al. Biophysical Constraints Arising from Compositional Context in Synthetic Gene Networks. Cell Syst 5, 11-24 e12, doi:10.1016/j.cels.2017.06.001 (2017).
34 Des Soye, B. J., Gerbasi, V. R., Thomas, P. M., Kelleher, N. L. & Jewett, M. C. A Highly Productive, One-Pot Cell-Free Protein Synthesis Platform Based on Genomically Recoded Escherichia coli. Cell Chem Biol 26, 1743-1754 e1749, doi:10.1016/j.chembiol.2019.10.008 (2019).
35 Caschera, F. et al. High-Throughput Optimization Cycle of a Cell-Free Ribosome Assembly and Protein Synthesis System. ACS Synth Biol 7, 2841-2853, doi:10.1021/acssynbio.8b00276 (2018).
36 Karim, A. S., Heggestad, J. T., Crowe, S. A. & Jewett, M. C. Controlling cell-free metabolism through physiochemical perturbations. Metab Eng 45, 86-94, doi:10.1016/j.ymben.2017.11.005 (2018).
37 Marshall, R., Garamella, J., Noireaux, V. & Pierson, A. High-throughput Microliter-Sized Cell-Free Transcription-Translation Reactions for Synthetic Biology Applications Using the Echo® 550 Liquid Handler. Labcyte Application Note, App-G124 (2018).
38 Densmore, D. M. & Bhatia, S. Bio-design automation: software+biology+robots. Trends Biotechnol 32, 111-113, doi:10.1016/j.tibtech.2013.10.005 (2014).
39 Hillson, N. J., Rosengarten, R. D. & Keasling, J. D. j5 DNA assembly design automation software. ACS Synth Biol 1, 14-21, doi:10.1021/sb2000116 (2012).
40 Hillson, N. J. j5 DNA assembly design automation. Methods Mol Biol 1116, 245-269, doi:10.1007/978-1-62703-764-8_17 (2014).
41 Xu, H., Liu, W. Q. & Li, J. Translation Related Factors Improve the Productivity of Streptomyces-Based Cell-Free Protein Synthesis System. ACS Synth Biol, doi:10.1021/acssynbio.0c00140 (2020).
42 Des Soye, B. J., Davidson, S. R., Weinstock, M. T., Gibson, D. G. & Jewett, M. C. Establishing a High-Yielding Cell-Free Protein Synthesis Platform Derived from Vibrio natriegens. ACS Synth Biol 7, 2245-2255, doi:10.1021/acssynbio.8b00252 (2018).
43 Wiegand, D. J., Lee, H. H., Ostrov, N. & Church, G. M. Establishing a Cell-Free Vibrio natriegens Expression System. ACS Synth Biol 7, 2475-2479, doi:10.1021/acssynbio.8b00222 (2018).
44 Minton, N. P. et al. A roadmap for gene system development in Clostridium. Anaerobe 41, 104-112, doi:10.1016/j.anaerobe.2016.05.011 (2016).

Supplemental Information

TABLE 1
DNA Vector Sequences. Below is a table of all
vectors used in this study. The vector type is
what is referenced throughout the manuscript.

| Vector Type | Vector Name | Vector DNA Sequence |
|---|---|---|
| pcExpress | pMTL8315_16_Pfer_TpepN | SEQ ID NO: 6 |
| pD1 | P8_pJLD1_Gene1 | SEQ ID NO: 1 |
| pD2 | pDN2_GG2_Pfer_GG3 | SEQ ID NO: 2 |
| pD3 | p14_pJLD2_Gene2 | SEQ ID NO: 3 |
| pD4 | pDN4_GG4_Pwl_GG5 | SEQ ID NO: 4 |
| pD5 | P1_pGLD3_Gene3 | SEQ ID NO: 5 |

TABLE 2
List of DNA Parts. Terminators, spacers, and
promoters are used to construct operons in pCExpress
vectors used in this study are listed.

| | Nucleotide sequence (5′ to 3′) |
|---|---|
| Terminators | |
| T1 (TgyrA) | SEQ ID NO: 7 |
| T2 (TtyrS) | SEQ ID NO: 8 |
| T3 (TpepN) | SEQ ID NO: 9 |
| Spacers synthesized by JGI | |
| pD1 variant 1 spacer region | SEQ ID NO: 10 |
| pD1 variant 2 spacer region | SEQ ID NO: 11 |
| pD1 variant 3 spacer region | SEQ ID NO: 12 |
| pD3 variant 1 spacer region | SEQ ID NO: 13 |
| pD3 variant 2 spacer region | SEQ ID NO: 14 |
| pD3 variant 3 spacer region | SEQ ID NO: 15 |
| pD5 variant 1 spacer region | SEQ ID NO: 16 |
| pD5 variant 2 spacer region | SEQ ID NO: 17 |
| pD5 variant 3 spacer region | SEQ ID NO: 18 |
| Promoters | |
| fdx | SEQ ID NO: 19 |
| pfor | SEQ ID NO: 20 |
| wl | SEQ ID NO: 21 |
| pta | SEQ ID NO: 22 |

TABLE 3
List of pCExpress and pD2/pD4 variants for assembly versatility. Each
variant is listed with name and backbone vector is derived from along
with which overhangs, promoter, and terminator is present.

| # | | Backbone | GG sites | Promoter | Terminator |
|---|---|---|---|---|---|
| | pCExpress vector name | | | | |
| 1 | pMTL8225_12_Pfer_TpepN | pMTL8225 | OV1, OV2 | fdx | TpepN |
| 2 | pMTL8225_14_Pfer_TpepN | pMTL8225 | OV1, OV4 | fdx | TpepN |
| 3 | pMTL8225_56_Pfer_TpepN | pMTL8225 | OV5, OV6 | fdx | TpepN |
| 4 | pMTL8225_36_Pfer_TpepN | pMTL8225 | OV3, OV6 | fdx | TpepN |
| 5 | pMTL8225_34_Pfer_TpepN | pMTL8225 | OV3, OV4 | fdx | TpepN |
| 6 | pMTL8225_12_Pwl_TpepN | pMTL8225 | OV1, OV2 | wl | TpepN |
| 7 | pMTL8225_14_Pwl_TpepN | pMTL8225 | OV1, OV4 | wl | TpepN |
| 8 | pMTL8225_56_Pwl_TpepN | pMTL8225 | OV5, OV6 | wl | TpepN |
| 9 | pMTL8225_36_Pwl_TpepN | pMTL8225 | OV3, OV6 | wl | TpepN |
| 10 | pMTL8225_34_Pwl_TpepN | pMTL8225 | OV3, OV4 | wl | TpepN |
| 11 | pMTL8225_12_Ppfor_TpepN | pMTL8225 | OV1, OV2 | pfor | TpepN |
| 12 | pMTL8225_14_Ppfor_TpepN | pMTL8225 | OV1, OV4 | pfor | TpepN |
| 13 | pMTL8225_56_Ppfor_TpepN | pMTL8225 | OV5, OV6 | pfor | TpepN |
| 14 | pMTL8225_36_Ppfor_TpepN | pMTL8225 | OV3, OV6 | pfor | TpepN |
| 15 | pMTL8225_34_Ppfor_TpepN | pMTL8225 | OV3, OV4 | pfor | TpepN |
| 16 | pMTL8225_12_Ppta_TpepN | pMTL8225 | OV1, OV2 | pta | TpepN |
| 17 | pMTL8225_14_Ppta_TpepN | pMTL8225 | OV1, OV4 | pta | TpepN |
| 18 | pMTL8225_56_Ppta_TpepN | pMTL8225 | OV5, OV6 | pta | TpepN |
| 19 | pMTL8225_36_Ppta_TpepN | pMTL8225 | OV3, OV6 | pta | TpepN |
| 20 | pMTL8225_34_Ppta_TpepN | pMTL8225 | OV3, OV4 | pta | TpepN |
| 21 | pMTL8315_12_Pfer_TpepN | pMTL8315 | OV1, OV2 | fdx | TpepN |
| 22 | pMTL8315_14_Pfer_TpepN | pMTL8315 | OV1, OV4 | fdx | TpepN |
| 23 | pMTL8315_56_Pfer_TpepN | pMTL8315 | OV5, OV6 | fdx | TpepN |
| 24 | pMTL8315_36_Pfer_TpepN | pMTL8315 | OV3, OV6 | fdx | TpepN |
| 25 | pMTL8315_34_Pfer_TpepN | pMTL8315 | OV3, OV4 | fdx | TpepN |
| 26 | pMTL8315_12_Pwl_TpepN | pMTL8315 | OV1, OV2 | wl | TpepN |
| 27 | pMTL8315_14_Pwl_TpepN | pMTL8315 | OV1, OV4 | wl | TpepN |
| 28 | pMTL8315_56_Pwl_TpepN | pMTL8315 | OV5, OV6 | wl | TpepN |
| 29 | pMTL8315_36_Pwl_TpepN | pMTL8315 | OV3, OV6 | wl | TpepN |
| 30 | pMTL8315_34_Pwl_TpepN | pMTL8315 | OV3, OV4 | wl | TpepN |
| 31 | pMTL8315_12_Ppfor_TpepN | pMTL8315 | OV1, OV2 | pfor | TpepN |
| 32 | pMTL8315_14_Ppfor_TpepN | pMTL8315 | OV1, OV4 | pfor | TpepN |
| 33 | pMTL8315_56_Ppfor_TpepN | pMTL8315 | OV5, OV6 | pfor | TpepN |
| 34 | pMTL8315_36_Ppfor_TpepN | pMTL8315 | OV3, OV6 | pfor | TpepN |
| 35 | pMTL8315_34_Ppfor_TpepN | pMTL8315 | OV3, OV4 | pfor | TpepN |
| 36 | pMTL8315_12_Ppta_TpepN | pMTL8315 | OV1, OV2 | pta | TpepN |
| 37 | pMTL8315_14_Ppta_TpepN | pMTL8315 | OV1, OV4 | pta | TpepN |
| 38 | pMTL8315_56_Ppta_TpepN | pMTL8315 | OV5, OV6 | pta | TpepN |
| 39 | pMTL8315_36_Ppta_TpepN | pMTL8315 | OV3, OV6 | pta | TpepN |
| 40 | pMTL8315_34_Ppta_TpepN | pMTL8315 | OV3, OV4 | pta | TpepN |
| 41 | pMTL8225_16_Pwl_TpepN | pMTL8225 | OV1, OV6 | wl | TpepN |
| 42 | pMTL8315_16_Pwl_TpepN | pMTL8315 | OV1, OV6 | wl | TpepN |
| 43 | pMTL8225_16_Pfer_TpepN | pMTL8225 | OV1, OV6 | fdx | TpepN |
| 44 | pMTL8225_16_Pwl_TpepN | pMTL8225 | OV1, OV6 | wl | TpepN |
| 45 | pMTL8225_16_Ppfor_TpepN | pMTL8225 | OV1, OV6 | pfor | TpepN |
| 46 | pMTL8225_16_Ppta_TpepN | pMTL8225 | OV1, OV6 | pta | TpepN |
| 47 | pMTL8315_16_Pfer_TpepN | pMTL8315 | OV1, OV6 | fdx | TpepN |
| 48 | pMTL8315_16_Pwl_TpepN | pMTL8315 | OV1, OV6 | wl | TpepN |
| 49 | pMTL8315_16_Ppfor_TpepN | pMTL8315 | OV1, OV6 | pfor | TpepN |
| 50 | pMTL8315_16_Ppta_TpepN | pMTL8315 | OV1, OV6 | pta | TpepN |
| | Donor vectors | | | | |
| 1 | pDN2_GG2_TtyrS_Pfer_GG3 | pD2 | OV2, OV3 | fdx | TtyrS |
| 2 | pDN2_GG2_TtyrS_Pwl_GG3 | pD2 | OV2, OV3 | wl | TtyrS |
| 3 | pDN2_GG2_TtyrS_Ppfor_GG3 | pD2 | OV2, OV3 | pfor | TtyrS |
| 4 | pDN4_GG4_TtyrS_Pfer_GG5 | pD4 | OV4, OV5 | fdx | TtyrS |
| 5 | pDN4_GG4_TtyrS_Pwl_GG5 | pD4 | OV4, OV5 | wl | TtyrS |
| 6 | pDN4_GG4_TtyrS_Ppfor_GG5 | pD4 | OV4, OV5 | pfor | TtyrS |
| 7 | pDN4_GG2_TtyrS_Pfer_GG5 | pD4 | OV4, OV5 | fdx | TtyrS |
| 8 | pDN4_GG2_TtyrS_Pwl_GG5 | pD4 | OV4, OV5 | wl | TtyrS |

TABLE 4
List of biosynthetic genes used CFE experiments.
Genes used are represented by their abbreviation,
organism, and Genbank accession numbers.

| | | |
|---|---|---|
| | | Genbank |
| Genes | Organism | accession # |

FIG. 2

| | | |
|---|---|---|
| ZraPhlA | Zoogloea ramigera | AAA27706.1 |
| CtyThlA | Clostridium tyrobutyricum | AND83415.1 |
| CacThlA1 | Clostridium acetobutylicum ATCC824 | AAA82724.1 |
| AveTer | Aeromonas veronii | WP_100652199.1 |
| EcoFadA | Escherichia coli | AAA23751.1 |
| LepFabV | Leptotrichia sp. | AP019845.1 |
| EcoFadB | Escherichia coli | AAA23750.1 |
| CnePhaB | Cupriavidus necator | AAA21973.1 |
| CsaHbd | Clostridium | WP_077360117.1 |
| | saccharoperbutylacetonicum | |
| CacHbd2 | Clostridium acetobutylicum ATCC824 | AAK80654.1 |
| CacHbd1 | Clostridium acetobutylicum ATCC824 | AAA95971.1 |
| AcaPhaJ | Aeromonas caviae | BAA21816.1 |
| PaePhaJ4 | Pseudomonas aeruginosa | BAC44835.1 |
| PaePhaJ1 | Pseudomonas aeruginosa | BAA92740.1 |
| CtyCrt | Clostridium tyrobutyricum | AND85881.1 |
| CacCrt | Clostridium acetobutylicum ATCC824 | AAA95967.1 |
| CbeHbd | Clostridium beijerinckii | AAM14586.1 |
| CauHbd1 | Clostridium autoethanogenum | AGY75809.1 |
| CauHbd2 | Clostridium autoethanogenum | WP_013240787.1 |
| CauHbd3 | Clostridium autoethanogenum | AGY74649.1 |
| CauHbd4 | Clostridium autoethanogenum | WP_122057754.1 |
| CauHbd5 | Clostridium autoethanogenum | AGY74784.1 |
| CauAdh1 | Clostridium autoethanogenum | AGY76060.1 |
| CauAdh2 | Clostridium autoethanogenum | AGY75962.1 |
| CauAdh3 | Clostridium autoethanogenum | AGY74883.1 |
| CauDgd | Clostridium autoethanogenum | AGY74614.1 |
| CauBdh | Clostridium autoethanogenum | AGY74782.1 |
| CnePhaB | Cupriavidus necator | AAA21973.1 |
| CneBktB | Cupriavidus necator | AAC38322.1 |
| EgrTer | Euglena gracilis | AAW66853.1 |
| CklHbd1 | Clostridium kluyveri | EDK32512.1 |
| CacCrt | Clostridium acetobutylicum ATCC824 | AAA95967.1 |
| CklThlA1 | Clostridium kluyveri | EDK35681.1 |

TABLE 5
List of biosynthetic genes used to assess codon
usage and assembly efficiency. Genes used are
represented by their Genbank accession numbers.
Biosynthetic genes (Genbank accession #)

5Z7R_A
AAD31841.1
OOP71501.1
WP_011785966.1
WP_134310305.1
WP_033987601.1
WP_034582189.1
WP_011967672.1
WP_077892378.1
WP_017751917.1
WP_140027439.1
AAB40248.1
AAA95971.1
4WYR_A
WP_012104014.1
WP_024243753.1
4W61_A
4W61_A (modified)
4N5L_A

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A system comprising one or more of the following components:
(a) a backbone vector for insertion of a donor sequence from one or more donor vectors, the backbone vector comprising from 5′→3′:
(i) a promoter for expressing a gene of interest in a cell;
(ii) a first Golden Gate site for cloning;
(iii) optionally a counter selectable marker;
(iv) a second Golden Gate site for cloning; and
(v) a transcription termination site;
(b) a first donor vector (pDonor1) for cell-free expression of a gene of interest, the pDonor1 comprising from 5′→3′:
(i) a promoter for cell-free RNA synthesis;
(ii) a first Golden Gate site for cloning;
(iii) optionally a first gene of interest (Gene1); and
(iv) a second Golden Gate site for cloning; wherein the optional Gene1 is inserted between the first Golden Gate site and the second Golden Gate site;
(c) a second donor vector (pDonor2) that comprises a donor promoter for use in expressing a gene of interest in a cell, the pDonor2 comprising from 5′→3′:
(i) a first Golden Gate site for cloning;
(ii) a transcription termination site;
(iii) a promoter for expressing a gene of interest in a cell; and
(iv) a second Golden Gate site for cloning; and
(d) a third donor vector (pDonor3) for cell-free expression of a gene of interest, the pDonor3 comprising from 5′→3′:
(i) a promoter for cell-free RNA synthesis;
(ii) a first Golden Gate site for cloning;
(iii) optionally a second gene of interest (Gene2); and
(iv) a second Golden Gate site for cloning; wherein the optional Gene2 is inserted between the first Golden Gate site and the second Golden Gate site.

2. The system of claim 1, wherein the system comprises two or more of the components: (a) the backbone vector, (b) the pDonor1, (c) the pDonor2, and (d) the pDonor3.

3. The system of claim 1, wherein the system comprises component (a) the backbone vector; and one or more of components (b) the pDonor1, (c) the pDonor2, and (d) the pDonor3.

4. The system of claim 1, wherein the system comprises components (a) the backbone vector, (b) the pDonor1, (c) the pDonor2, and (d) the pDonor3.

5. The system of claim 1, wherein the system comprises as components:
(a) the backbone vector for insertion of a donor sequence from a donor vector, the backbone vector comprising from 5′→3′:
(i) a first promoter for expressing a gene of interest in a cell (P1);
(ii) a first Golden Gate site for cloning (GG1);
(iii) optionally a counter selectable marker;
(iv) a second Golden Gate site for cloning (GG2); and
(v) a transcription termination site (TT);
(b) the first donor vector (pDonor1) for cell-free expression of a gene of interest, the pDonor1 comprising from 5′→3′:
(i) a promoter for cell-free RNA synthesis;
(ii) a first Golden Gate site for cloning (GG1);
(iii) optionally a first gene of interest (Gene1); and
(iv) a second Golden Gate site for cloning (GG2); wherein the optional Gene1 is inserted between GG1 and GG2.

6. The system of claim 1, wherein the system comprises as components:
(a) the backbone vector for insertion of donor sequences from one or more donor vectors, the backbone vector comprising from 5′→3′:
(i) a first promoter for expressing a gene of interest in a cell (P1);
(ii) a first Golden Gate site for cloning (GG1);
(iii) optionally a counter selectable marker;
(iv) a terminal Golden Gate site for cloning (GGT); and
(v) a transcription termination site (TT);
(b) the first donor vector (pDonor1) for cell-free expression of a gene of interest, the pDonor1 comprising from 5′→3′:
(i) a promoter for cell-free RNA synthesis;
(ii) a first Golden Gate site for cloning (GG1);
(iii) optionally a first gene of interest (Gene1); and
(iv) a second Golden Gate site for cloning (GG2); wherein the optional Gene1 is inserted between GG1 and GG2.
(c) a second donor vector (pDonor2) that comprises a donor promoter for use in expressing a gene of interest in a cell, pDonor2 comprising from 5′→3′:
(i) a second Golden Gate site for cloning (GG2);
(ii) a first transcription termination site (T1);
(iii) a second promoter for expressing a gene of interest in a cell (P2); and
(iv) a third Golden Gate site for cloning (GG3); and
(d) a third donor vector (pDonor3) for cell-free expression of a gene of interest, pDonor3 comprising from 5′→3′:
(i) a promoter for cell-free RNA synthesis;
(ii) a third Golden Gate site for cloning (GG3);
(iii) optionally a second gene of interest (Gene2); and
(iv) a terminal Golden Gate site for cloning (GGT); wherein the optional Gene2 is inserted between GG3 and GGT.

7. The system of claim 1, wherein the system comprises as components:
(a) the backbone vector for insertion of donor sequences from one or more donor vectors, the backbone vector comprising from 5′→3′:
(i) a first promoter for expressing a gene of interest in a cell (P1);
(ii) a first Golden Gate site for cloning (GG1);
(iii) optionally a counter selectable marker;
(iv) a terminal Golden Gate site for cloning (GGT); and
(v) a terminal transcription termination site (TT);
(b) the first donor vector (pDonor1) for cell-free expression of a gene of interest, pDonor1 comprising from 5′→3′:
(i) a promoter for cell-free RNA synthesis;
(ii) a first Golden Gate site for cloning (GG1);
(iii) optionally a first gene of interest (Gene1); and
(iv) a second Golden Gate site for cloning (GG2); where the optional Gene1 is inserted between GG1 and GG2;
(c) the second donor vector (pDonor2) that comprises a donor promoter for use in expressing a gene of interest in a cell, pDonor2 comprising from 5′→3′:
(i) a second Golden Gate site for cloning (GG2);
(ii) a first transcription termination site (T1);
(iii) a second promoter for expressing a gene of interest in a cell (P2); and
(iv) a third Golden Gate site for cloning (GG3); and
(d) the third donor vector (pDonor3) for cell-free expression of a gene of interest, pDonor3 comprising from 5′→3′:
(i) a promoter for cell-free RNA synthesis;
(ii) a third Golden Gate site for cloning (GG3);
(iii) optionally a second gene of interest (Gene2); and
(iv) a fourth Golden Gate site for cloning (GG4); where the optional Gene2 is inserted between GG3 and GG4;
(e) a fourth donor vector (pDonor4) that comprises a donor promoter for use in expressing a gene of interest in a cell, pDonor4 comprising from 5′→3′:
(i) a fourth Golden Gate site for cloning (GG4);
(ii) a second transcription termination site (T2);
(iii) a third promoter for expressing a gene of interest in a cell (P3); and
(iv) a fifth Golden Gate site for cloning (GG5); and
(f) a fifth donor vector (pDonor5) for cell-free expression of a gene of interest, pDonor5 comprising from 5′→3′:
(i) a promoter for cell-free RNA synthesis;
(ii) a fifth Golden Gate site for cloning (GG5);
(iii) optionally a third gene of interest (Gene3); and
(iv) a terminal Golden Gate site for cloning (GGT); where the optional Gene3 is inserted between GG5 and GGT.

8. The system of claim 5, wherein pDonor1 comprises Gene1, and optionally Gene1 has been codon-optimized for expression in a cell-free system or for expression in a cell.

9. The system of claim 6, wherein pDonor1 and pDonor3 comprise Gene1 and, Gene2, respectively, and optionally Gene1 and Gene2 have been codon-optimized for expression in a cell-free system or for expression in a cell.

10. The system of claim 7, wherein pDonor1, pDonor3, pDonor5 comprise Gene1, Gene2, and Gene3 respectively, and Gene1, Gene2, and Gene3 have been codon-optimized for expression in a cell-free system or for expression in a cell.

11. The system of claim 8, wherein Gene1 has been codon-optimized for expression in a cell-free system comprising a cellular lysate from Clostridia, or wherein Gene1 has been codon-optimized for expression in a Clostridia cell.

12. The system of claim 9, wherein Gene1 and Gene2 have been codon-optimized for expression in a cell-free system comprising a cellular lysate from Clostridia, or wherein Gene1 and Gene2 have been codon-optimized for expression in a Clostridia cell.

13. The system of claim 10, wherein Gene1, Gene2, and Gene3 have been codon-optimized for expression in a cell-free system comprising a cellular lysate from Clostridia, or wherein Gene1, Gene2, and Gene3 have been codon-optimized for expression in a Clostridia cell.

14. The system of claim 6, wherein pDonor2 comprises a promoter that has been engineered to express a gene in Clostridia or in a cell-free extract from Clostridia.

15. The system of claim 7, wherein pDonor2 and pDonor4 comprise a promoter that has been engineered to express a gene in Clostridia or in a cell-free extract from Clostridia.

16. The system of claim 5, wherein the first Golden Gate site for cloning (GG1) and the second Golden Gate site for cloning (GG2) are the same or different and comprise a recognition site for a TypeIIS restriction enzyme optionally oriented so as to cleave upstream (5′) of its recognition site and provide an overhang that hybridizes to its reverse complement overhang.

17. The system of claim 6, wherein the first Golden Gate site for cloning (GG1), the second Golden Gate site for cloning (GG2), the third Golden Gate site for cloning (GG3), and the terminal Golden Gate for cloning (GGT) are the same or different and comprise a recognition site for a TypeIIS restriction enzyme optionally oriented so as to cleave upstream (5′) of its recognition site and provide an overhang that hybridizes to its reverse complement overhang.

18. The system of claim 7, wherein the first Golden Gate site for cloning (GG1), the second Golden Gate site for cloning (GG2), the third Golden Gate site for cloning (GG3), the fourth Golden Gate for cloning (GG4), the fifth Golden Gate site for cloning (GG5, and the terminal Golden Gate for cloning (GGT) are the same or different and comprise a recognition site for a TypeIIS restriction enzyme optionally oriented so as to cleave upstream (5′) of its recognition site and provide an overhang that hybridizes to its reverse complement overhang.

19. The system of claim 16, wherein the TypeIIS restriction enzyme is selected from a BsaI site, a BbsI site, and an AarI site

20. The system of claim 17, wherein the TypeIIS restriction enzyme is selected from a BsaI site, a BbsI site, and an AarI site.

21. The system of claim 18, wherein the TypeIIS restriction enzyme is selected from a BsaI site, a BbsI site, and an AarI site.

22. The system of claim 5, wherein the promoter for cell-free RNA synthesis of the first donor vector is a promoter for a bacteriophage DNA-dependent RNA polymerase.

23. The system of claim 6, wherein the promoter for cell-free RNA synthesis of one or more of the first donor vector and the third donor vector is a promoter for a bacteriophage DNA-dependent RNA polymerase.

24. The system of claim 7, wherein the promoter for cell-free RNA synthesis of one or more of the first donor vector, the third donor vector, and the fifth donor vector is a promoter for a bacteriophage DNA-dependent RNA polymerase.

25. The system of claim 5, wherein pDonor1 comprises the polynucleotide sequence of SEQ ID NO:24, SEQ ID NO:27; and/or SEQ ID NO:30.

26. The system of claim 6, wherein pDonor1 comprises the polynucleotide sequence of SEQ ID NO:24, SEQ ID NO:27; and/or SEQ ID NO:30 and/or pDonor3 comprises the polynucleotide sequence of SEQ ID NO:25, SEQ ID NO:28, or SEQ ID NO:31.

27. The system of claim 7, wherein pDonor1 comprises the polynucleotide sequence of SEQ ID NO:24, SEQ ID NO:27, or SEQ ID NO:30; and/or pDonor3 comprises the polynucleotide sequence of SEQ ID NO:25, SEQ ID NO:28, or SEQ ID NO:31; and/or pDonor5 comprises the polynucleotide sequence of SEQ ID NO:26, SEQ ID NO:29, or SEQ ID NO:32.

28. A cell transformed with the system of claim 5.

29. A cell transformed with the system of claim 6.

30. A cell transformed with the system of claim 7.

31. A method for expressing a gene of interest, such as Gene1, the method comprising cloning the gene of interest into a vector of the system of claim 5 and expressing the gene of interest in a cell-free system or in a cell.

32. A method for expressing a gene of interest, such as Gene1 or Gene2, the method comprising cloning the gene of interest into a vector of the system of claim 6 and expressing the gene of interest in a cell-free system or in a cell.

33. A method for expressing a gene of interest comprising Gene1, Gene2, or Gene3, the method comprising cloning the gene of interest into a vector of the system of claim 7 and expressing the gene of interest in a cell-free system or in a cell.

34. A method for expressing multiple genes of interest in a cell comprising Gene1 and Gene2, the method comprising cloning the multiple genes of interest into one or more vectors of the system of claim 6, further cloning the multiple genes of interest into the backbone vector of the system of claim 6, introducing the backbone vector into a cell, and expressing the multiple genes of interest in the cell.

35. The method of claim 34, wherein the multiple genes of interest are expressed from multiple different promoters.

36. A method for expressing multiple genes of interest in a cell comprising Gene1, Gene2, or Gene3, the method comprising cloning the multiple genes of interest into one or more vectors of the system of claim 7, further cloning the multiple genes of interest into the backbone vector of claim 8, introducing the backbone vector into a cell, and expressing the multiple genes of interest in the cell.

37. The method of claim 36, wherein the multiple genes of interest are expressed from multiple different promoters.

38. A method for selecting genes for expression in a cell, the method comprising one or more of the following steps:
(a) cloning the genes into a vector of the system of claim 5, 6, or 7;
(b) testing expression of the genes in a cell-free expression system;
(c) selecting genes that are expressed in the cell-free expression system;
(d) cloning the selecting genes into a Clostridium expression vector; and
(e) transforming Clostridium with the expression vector and test expression of the genes in Clostridium

39. A polynucleotide comprising the polynucleotide sequence of any of SEQ ID NOs:1-32.

40. A combination of two or more separate polynucleotides, each of the two or more separate polynucleotides comprising the polynucleotide sequence of any of SEQ ID NOs:1-32.
